SEQUENCE LISTING (58 sequences)

SEQ ID NO: 1 - protein, 217 aa, synthetic construct
  source 1..217: note = Human IgG1 Fc sequence 231-447 (EU-numbering)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 607 aa, synthetic construct
  source 1..607: note = Amino Acid Sequence of Human HER2
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPIN

SEQ ID NO: 3 - protein, 448 aa, synthetic construct
  source 1..448: note = Clone ID 3468 Full
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKGYFPEPVTVSWNSGALTSGVHTFPAVLKSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 4 - protein, 119 aa, synthetic construct [sequence omitted]

SEQ ID NO: 5 - protein, 8 aa, synthetic construct
  source 1..8: note = Clone ID 3468,3057,3041,3317 CDRH1
GFTFTDYT

SEQ ID NO: 6 - protein, 12 aa, synthetic construct
  source 1..12: note = Clone ID 3468,3057,3041,3317 H3
ARNLGPSFYFDY

SEQ ID NO: 7 - protein, 8 aa, synthetic construct
  source 1..8: note = Clone ID 3468,3057,3041,3317 H2
VNPNSGGS

SEQ ID NO: 8 - protein, 214 aa, synthetic construct
  source 1..214: note = Clone ID 1811 Full
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 107 aa, synthetic construct [sequence omitted]

SEQ ID NO: 10 - protein, 6 aa, synthetic construct
  source 1..6: note = Clone ID 1811, 3904, 3317 CDRL1
QDVSIG

SEQ ID NO: 11 - protein, 9 aa, synthetic construct
  source 1..9: note = Clone ID 1811, 3904, 3317 CDRL3
QQYYIYPYT

SEQ ID NO: 12 - skipped

SEQ ID NO: 13 - protein, 222 aa, synthetic construct
  source 1..222: note = Clone ID 5034 Full
DYKDDDDKDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDERLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 107 aa, synthetic construct [sequence omitted]

SEQ ID NO: 15 - protein, 222 aa, synthetic construct
  source 1..222: note = Clone ID 5037 Full
DYKDDDDKDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDERLKSGTASVVCLLNNFYPREAKVQWKVDNALQSGNSKESVTEQDSKDSTYSLSSRLTLSKADYEKHKVYACEVTHQGLSSPVTKSFNRGEC

SEQ ID NO: 16 - protein, 107 aa, synthetic construct [sequence omitted]

SEQ ID NO: 17 - protein, 6 aa, synthetic construct
  source 1..6: note = Clone ID 5037 CDRL1
QDVNTA

SEQ ID NO: 18 - protein, 9 aa, synthetic construct
  source 1..9: note = Clone ID 5037 CDRL3
QQHYTTPPT

SEQ ID NO: 19 - skipped

SEQ ID NO: 20 - protein, 215 aa, synthetic construct
  source 1..215: note = Clone ID 3382 Full
GDIQMTQSPSSLSASVGDRVTITCKASQDVSIGVAWYQQKPGKAPKLLIYSASYRYTGVPSRFSGSGSGTDFTLTISSLQPEDFATYYCQQYYIYPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 107 aa, synthetic construct
  source 1..107: note = Clone ID 3382 VL
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPATFGQGTKVEIK

SEQ ID NO: 22 - protein, 6 aa, synthetic construct
  source 1..6: note = Clone ID 3382 CDRL1
QDVSIG

SEQ ID NO: 23 - protein, 9 aa, synthetic construct
  source 1..9: note = Clone ID 3382 CDRL3
QQYYIYPAT

SEQ ID NO: 24 - skipped

SEQ ID NO: 25 - protein, 449 aa, synthetic construct
  source 1..449: note = Clone ID 5065 Full
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCEVTDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 26 - protein, 120 aa, synthetic construct [sequence omitted]

SEQ ID NO: 27 - protein, 8 aa, synthetic construct
  source 1..8: note = Clone ID 5065, 720, 719 CDRH1
GFNIKDTY

SEQ ID NO: 28 - protein, 13 aa, synthetic construct
  source 1..13: note = Clone ID 5065, 720, 719 H3
SRWGGDGFYAMDY

SEQ ID NO: 29 - protein, 8 aa, synthetic construct
  source 1..8: note = Clone ID 5065, 720, 719 H2
IYPTNGYT

SEQ ID NO: 30 - protein, 449 aa, synthetic construct
  source 1..449: note = Clone ID 6586 Full
GEVQLVESGGGLVQPGGSLRLSCAASGFTFADYTMDWVRQAPGKGLEWVGDVNPNSGGSIYNQRFKGRFTFSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 31 - protein, 119 aa, synthetic construct
  source 1..119: note = Clone ID 6586 VH
EVQLVESGGGLVQPGGSLRLSCAASGFTFADYTMDWVRQAPGKGLEWVGDVNPNSGGSIYNQRFKGRFTFSVDRSKNTLYLQMNSLRAEDTAVYYCARNLGPSFYFDYWGQGTLVTVSS

SEQ ID NO: 32 - protein, 8 aa, synthetic construct
  source 1..8: note = Clone ID 6586 CDRH1
GFTFADYT

SEQ ID NO: 33 - protein, 12 aa, synthetic construct
  source 1..12: note = Clone ID 6586 H3
ARNLGPSFYFDY

SEQ ID NO: 34 - protein, 8 aa, synthetic construct
  source 1..8: note = Clone ID 6586 H2
VNPNSGGS

SEQ ID NO: 35 - protein, 226 aa, synthetic construct
  source 1..226: note = Clone ID 3904 Full
YPYDVPDYATGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEELKSGTASVVCLLNNFYPREAKVQWKVDNALQSGNSEESVTEQDSKDSTYSLSSTLELSKADYEKHKVYACEVTHQGLSSPVTKSFNRGEC

SEQ ID NO: 36 - protein, 107 aa, synthetic construct [sequence omitted]

SEQ ID NO: 37 - protein, 481 aa, synthetic construct
  source 1..481: note = Clone ID 719 Full
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDEDGSFALVSKLTVDKSRWQQGNVFSCSVMHEALHNHYTQKSLSLSPGK

SEQ ID NO: 38 - protein, 107 aa, synthetic construct [sequence omitted]

SEQ ID NO: 39 - protein, 120 aa, synthetic construct [sequence omitted]

SEQ ID NO: 40 - protein, 481 aa, synthetic construct
  source 1..481: note = Clone ID 720 Full
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLICLVKGFYPSDIAVEWESNGQPENRYMTWPPVLDSDGSFFLYSKLTVDKSRWQQGNVFSCSVMHEALHNHYTQKSLSLSPGK

SEQ ID NO: 41 - protein, 107 aa, synthetic construct [sequence omitted]

SEQ ID NO: 42 - protein, 120 aa, synthetic construct [sequence omitted]

SEQ ID NO: 43 - protein, 448 aa, synthetic construct
  source 1..448: note = Clone ID 3041 Full
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 44 - protein, 119 aa, synthetic construct [sequence omitted]

SEQ ID NO: 45 - protein, 448 aa, synthetic construct
  source 1..448: note = Clone ID 3057 Full
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 46 - protein, 119 aa, synthetic construct [sequence omitted]

SEQ ID NO: 47 - protein, 475 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 48 - protein, 107 aa, synthetic construct [sequence omitted]

SEQ ID NO: 49 - protein, 119 aa, synthetic construct [sequence omitted]

SEQ ID NO: 50 - protein, 481 aa, synthetic construct
  source 1..481: note = Clone ID 5244 Full
GDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 51 - protein, 107 aa, synthetic construct [sequence omitted]

SEQ ID NO: 52 - protein, 120 aa, synthetic construct [sequence omitted]

SEQ ID NO: 53 - protein, 6 aa, synthetic construct
  source 1..6: note = Clone ID 5244, 5034, 719, 720 CDRL1
QDVNTA

SEQ ID NO: 54 - skipped

SEQ ID NO: 55 - protein, 9 aa, synthetic construct
  source 1..9: note = Clone ID 5244, 5034, 719, 720 CDRL3
QQHYTTPPT

SEQ ID NO: 56 - protein, 8 aa, synthetic construct
  source 1..8: note = Clone ID 5422 CDRH1
GFNIKDTY

SEQ ID NO: 57 - protein, 8 aa, synthetic construct
  source 1..8: note = Clone ID 5244 H2
IYPTNGYT

SEQ ID NO: 58 - protein, 13 aa, synthetic construct
  source 1..13: note = Clone ID 5244 H3
SRWGGDGFYAMDY

(19) United States
(12) Patent Application Publication
(10) Pub. No.: US 2025/0136714 A1
(43) Pub. Date: May 1, 2025

(54) METHODS OF TREATING CANCER WITH ANTI-HER2 BIPARATOPIC ANTIBODIES

(71) Applicant: Zymeworks BC Inc., Vancouver (CA)

(72) Inventors: Rupert H. Davies, Seattle, WA (US); Neil C. Josephson, Seattle, WA (US); Jeffrey Ryan Proctor, Vancouver (CA)

(21) Appl. No.: 18/692,278

(22) PCT Filed: Sep. 15, 2022
(86) PCT No.: PCT/CA2022/051375

§ 371 Date: Mar. 14, 2024

Related U.S. Application Data
(60) Provisional application No. 63/244,690, filed on Sep. 15, 2021.

(51) Int. Cl. C07K 16/32; A61K 31/337; A61K 31/513; A61K 31/519; A61K 31/555; A61K 31/7068; A61K 33/243; A61K 39/00; A61P 35/00; C07K 16/28

(52) U.S. Cl. CPC C07K 16/32; A61K 31/337; A61K 31/513; A61K 31/519; A61K 31/555; A61K 31/7068; A61K 33/243; A61P 35/00; C07K 16/2818; A61K 2039/505; A61K 2039/545; C07K 2317/31; C07K 2317/55; C07K 2317/565; C07K 2317/622

(57) ABSTRACT

Methods of treating subjects having a HER2-expressing cancer with an anti-HER2 biparatopic antibody using a 2-tiered fixed dosing regimen based on the weight of subjects being treated are described. Combination therapy with chemotherapeutic agents and/or a PD-1 inhibitor, for example an anti-PD-1 antibody, are also described.

### FIELD

The present disclosure relates to the field of cancer therapeutics and, in particular, to dosing regimens for use in treating cancer with biparatopic anti-HER2 antibodies.

### BACKGROUND

HER2 (ErbB2) is a transmembrane surface-bound receptor tyrosine kinase that is a member of the ErbB family of receptor tyrosine kinases and is normally involved in the signal transduction pathways leading to cell growth and differentiation. HER2 is a promising target for treatment of breast cancer as it was found to be overexpressed in about one-quarter of breast cancer patients (Bange et al, Nature Medicine 7:548 (2001)).

Herceptin® (trastuzumab, U.S. Pat. No. 5,821,337) was the first monoclonal antibody developed for the treatment of HER2-positive breast cancer and has increased survival times for patients so that they are now the same as for patients with HER2-negative breast cancer. Pertuzumab (Perjeta®, U.S. Pat. No. 7,862,817) is a humanized monoclonal antibody, which is designed specifically to prevent the HER2 receptor from pairing (dimerizing) with other HER receptors (EGFR/HER1, HER3 and HER4) on the surface of cells, a process that is believed to play a role in tumor growth and survival. The combination of Perjeta, Herceptin and chemotherapy is thought to provide a more comprehensive blockade of HER signaling pathways. Pertuzumab binds to domain II of HER2, essential for dimerization, while trastuzumab binds to extracellular domain IV of HER2.

Li et al (Cancer Res., 73:6471-6483 (2013)) describe bispecific, bivalent antibodies to HER2 that are based on the native trastuzumab and pertuzumab sequences and which overcome trastuzumab resistance. Other bispecific anti-HER2 antibodies have been described (International Patent Application Publication Nos. WO 2015/077891 and WO 2016/179707; U.S. Patent Application Publication Nos. 2014/0170148, 2015/0284463, 2017/0029529 and 2017/0291955; U.S. Pat. No. 9,745,382). International Patent Application Publication No. WO 2016/082044 describes dosing regimens for anti-HER2 biparatopic antibodies.

International Patent Application Publication No. WO 2019/173911 describes anti-HER2 biparatopic antibody-drug conjugates comprising an auristatin analogue.

Most marketed antibody-based therapeutics are administered to subjects in dosages based either on the weight or the body surface area of a subject. For example, a therapeutic antibody may be administered at a dosage of X mg/kg of body weight, or Y mg/m² of body surface area. For example, the monoclonal antibody panitumumab has been approved has been approved for administration at a dosage of 6 mg/kg every 2 weeks (Q2W) and the monoclonal antibody nivolumab has been approved for administration at a dosage of 3 mg/kg Q2W. The monoclonal antibody rituximab has been approved for administration at a dosage of 375 mg/m² body surface area. The monoclonal antibody cetuximab has been approved for administration at a dosage of 250 mg/m² body surface area every week after a loading dose of 400 mg/m². Recently, the administration of therapeutic antibodies at fixed dosages (independent of body weight or body surface area) has been suggested (Hendrikx, J. et al., Fixed Dosing of Monoclonal Antibodies in Oncology, The Oncologist 22:1212 (2017)).

This background information is provided for the purpose of making known information believed by the applicant to be of possible relevance to the present disclosure. No admission is necessarily intended, nor should be construed, that any of the preceding information constitutes prior art against the claimed invention.

### SUMMARY

Described herein are methods of treating cancer using anti-HER2 biparatopic antibodies. In one aspect, the present disclosure relates to a method of treating a subject having a HER2-expressing cancer comprising administering to the subject an effective amount of an anti-HER2 biparatopic antibody at a fixed time interval, the effective amount comprising a fixed dose of the antibody administered.

In another aspect, the present disclosure relates to a method of treating a subject having a HER2-expressing cancer comprising administering to the subject, at a fixed time interval, an effective amount of an anti-HER2 biparatopic antibody, the effective amount comprising a two-tiered fixed dose, wherein a low fixed dose is administered to a subject weighing less than a weight cutoff point, and a high fixed dose is administered to a subject weighing at or above the weight cutoff point.

In another aspect, the present disclosure relates to a method of treating a subject having a HER2-expressing cancer comprising administering to the subject, at a fixed time interval, an effective amount of an anti-HER2 biparatopic antibody, the effective amount comprising a two-tiered fixed dose, wherein a low fixed dose is administered to a subject weighing less than 70 kg, and a high fixed dose is administered to a subject weighing 70 kg or more.

In another aspect, the present disclosure relates to a method of treating a subject having a HER2-expressing cancer comprising administering to the subject an effective amount of an anti-HER2 biparatopic antibody, the effective amount comprising a fixed dose of 1800 mg to a subject weighing less than 70 kg, or a fixed dose of 2400 mg to a subject weighing 70 kg or more, wherein the dose is administered every 3 weeks (Q3W).

In another aspect, the present disclosure relates to a method of treating a subject having a HER2-expressing cancer comprising administering to the subject an effective amount of an anti-HER2 biparatopic antibody, the effective amount comprising a fixed dose of 1800 mg to a subject weighing less than 70 kg, or a fixed dose of 2400 mg to a subject weighing 70 kg or more, wherein the dose is administered every 3 weeks (Q3W) wherein the HER2 biparatopic antibody comprises (a) a first antigen-binding domain comprising a variable heavy chain region (VH) comprising the sequence as set forth in SEQ ID NO: 31, and a variable light chain region (VL) comprising the sequence as set forth in SEQ ID NO: 21, and (b) a second antigen-binding domain comprising a VH sequence as set forth in SEQ ID NO: 52, and a VL sequence as set forth in SEQ ID NO: 51.

In another aspect, the present disclosure relates to a method of treating a subject having a HER2-expressing cancer comprising administering to the subject an effective amount of an anti-HER2 biparatopic antibody, the effective amount comprising a fixed dose of 1800 mg to a subject weighing less than 70 kg, or a fixed dose of 2400 mg to a subject weighing 70 kg or more, wherein the dose is administered every 3 weeks (Q3W), wherein the subject has been diagnosed with breast cancer, gastroesophageal adenocarcinoma (GEA), esophageal cancer, gastric cancer, endometrial cancer, ovarian cancer, cervical cancer, non-small cell lung cancer (NSCLC), anal cancer, colorectal cancer (CRC) or biliary tract cancer.

In another aspect, the present disclosure relates to a method of treating a subject having a HER2-expressing cancer comprising administering to the subject an effective amount of an anti-HER2 biparatopic antibody, the effective amount comprising a fixed dose of 1800 mg to a subject weighing less than 70 kg, or a fixed dose of 2400 mg to a subject weighing 70 kg or more, wherein the dose is administered every 3 weeks (Q3W), wherein the subject has been diagnosed with gastroesophageal adenocarcinoma (GEA), esophageal cancer, gastroesophageal junction cancer (GEJ), or gastric cancer.

In another aspect, the present disclosure relates to a method of treating a subject having a HER2-expressing cancer comprising administering to the subject an effective amount of an anti-HER2 biparatopic antibody, the effective amount comprising a fixed dose of 1200 mg to a subject weighing less than 70 kg, or a fixed dose of 1600 mg to a subject weighing 70 kg or more, wherein the dose is administered every 2 weeks (Q2W).

In another aspect, the present disclosure relates to a method of treating a subject having a HER2-expressing cancer comprising administering to the subject an effective amount of an anti-HER2 biparatopic antibody, the effective amount comprising a fixed dose of 1200 mg to a subject weighing less than 70 kg, or a fixed dose of 1600 mg to a subject weighing 70 kg or more, wherein the dose is administered every 2 weeks (Q2W), wherein the subject has been diagnosed with biliary tract cancer.

In another aspect, the present disclosure relates to a method of treating a subject having a HER2-expressing cancer comprising administering to the subject an effective amount of an anti-HER2 biparatopic antibody, the effective amount comprising a fixed dose of 1200 mg to a subject weighing less than 70 kg, or a fixed dose of 1600 mg to a subject weighing 70 kg or more, wherein the dose is administered every 2 weeks (Q2W) wherein the HER2 biparatopic antibody comprises (a) a first antigen-binding domain comprising a variable heavy chain region (VH) comprising the sequence as set forth in SEQ ID NO: 31, and a variable light chain region (VL) comprising the sequence as set forth in SEQ ID NO: 21, and (b) a second antigen-binding domain comprising a VH sequence as set forth in SEQ ID NO: 52, and a VL sequence as set forth in SEQ ID NO: 51. In another aspect, the present disclosure relates to a a pharmaceutical kit comprising: (i) one or more containers comprising an anti-HER-2 biparatopic antibody and (ii) a label or package insert in or associated with the one or more containers indicating that the anti-HER2 biparatopic antibody is for administration to a subject having a HER2-expressing cancer (a) at a dose of 1800 mg for a subject weighting less than 70 kg or (b) at a dose of 2400 mg for a subject weighing 70 kg or more, administered every 3 weeks (Q3W).

In another aspect, the present disclosure relates to a pharmaceutical kit comprising an anti-HER-2 biparatopic antibody and (ii) a label or package insert in or associated with the one or more containers indicating that the anti-HER2 biparatopic antibody is for administration to a subject having a HER2-expressing cancer (a) at a dose of 1200 mg for a subject weighting less than 70 kg or (b) at a dose of 1600 mg for a subject weighing 70 kg or more, administered every 2 weeks (Q2W).

### BRIEF DESCRIPTION OF THE DRAWINGS

FIG. 1 shows a schematic representation of the anti-HER2 biparatopic antibody v10000.

FIG. 2 shows a visual predictive check of median predicted exposure (solid line), 95% prediction interval (grey band) compared to observed data (open circles), median observed concentration (dotted line), and observed 2.5th/97.5th percentiles (dashed lines) for various dosing regimens. FIG. 2A, dose of 5 mg/kg administered weekly (QW). FIG. 2B, dose of 10 mg/kg administered weekly (Q2W). FIG. 2C, dose of 20 mg/kg administered every 2 weeks (Q2W).

FIG. 2D, dose of 30 mg/kg administered every 3 weeks (Q3W).

FIG. 3 shows model-predicted AUC at steady state by body weight using several dosing regimens. FIG. 3A, weight-based (30 mg/kg Q3W) dosing. FIG. 3B, one-tiered (2100 mg Q3W) flat dosing; FIG. 3C, two-tiered flat dosing (1800/2400 mg Q3W). The two-tiered flat dose (1800/2400 mg Q3W) is administered as 1800 mg to patients below 70 kg, and 2400 mg to patients above 70 kg.

FIG. 4 is a schematic showing the design of the clinical study described in Example 4 of v10000 in the treatment of gastrointestinal cancers. 5-FU=5-fluorouracil; DCR=disease control rate; DOR=duration of response; ECOG PS=Eastern Cooperative Oncology Group performance status; FISH=fluorescence in situ hybridization; GEA=gastroesophageal adenocarcinoma; IHC=immunohistochemistry; ORR=objective response rate; PD=progressive disease; PFS=progression-free survival; RECIST v1.1=Response Evaluation Criteria in Solid Tumors, version 1.1; SD=stable disease.

FIG. 5 is a waterfall plot showing the change in target size lesions in subjects being treated in the with the v10000 and one of the chemotherapy regimens CAPOX, FP or mFOLFOX. 5-FU=5-fluorouracil; CA=primary tumor location; CAPOX=capecitabine plus oxaliplatin; E=esophageal cancer; F=flat dosing; FISH=fluorescence in situ hybridization; FP=5-FU plus cisplatin; G=gastric cancer; IHC=immunohistochemistry; J=gastroesophageal junction cancer; mFOLFOX6=5-FU plus oxaliplatin and leucovorin; W=weight-based dosing; ZDR=2-tiered flat dosing regimen.

### DETAILED DESCRIPTION

The present disclosure relates to methods of treating a HER2-expressing cancer with an anti-HER2 biparatopic antibody. Most antibody-based therapeutics are administered to subjects in dosages based either on the weight (kg) or the body surface area (m²) of a subject. However, this method of dosing is inconvenient, because a specific amount must be calculated and dispensed for each patient. It also leads to drug wastage, since some subjects require more drug than others, and the drug usually is packaged in one or two uniform vial sizes. Unused drug in a vial often must be discarded. Therapeutic antibodies are expensive to manufacture, and wastage of drug is costly. To avoid these issues, some antibody manufacturers have developed a “one size fits all” or fixed dose of a therapeutic antibody that can be used for all patients independent of body weight or body surface area. However, this approach can lead to non-uniformity in the drug concentration within the subject, with some subjects having significantly more drug exposure than others. Using population pharmacokinetics, we have developed a tiered fixed dosing method wherein subjects below a certain weight are given a fixed dose that is lower than the fixed dose given to heavier subjects.

Thus in certain aspects of the methods disclosed herein, an anti-HER2 biparatopic antibody is administered to a subject having a HER2-expressing cancer in accordance with a two-tiered fixed dosing regimen depending on the weight of the subject and at a dosing interval fixed at every one week (QW), every 2 weeks (Q2W) or every 3 weeks (Q3W). In certain embodiments, the anti-HER2 biparatopic antibody is administered to the subject at a dose of about 1800 mg (for subjects<70 kg) or about 2400 mg (for subjects≥70 kg) IV Q3W on Day 1 of each 21-day cycle. In certain embodiments, the anti-HER2 biparatopic antibody is administered to the subject at a dose of about 1200 mg (for subjects<70 kg) or about 1600 mg (for subjects≥70 kg) IV Q2W on Days 1 and 15 of each 28-day cycle.

In certain aspects of the methods disclosed herein, the anti-HER2 biparatopic antibody is administered in combination with a chemotherapeutic agent. In certain aspects of the method of the present disclosure, the anti-HER2 antibody is administered with a PD-1 inhibitor, for example, an anti-PD-1 antibody.

### Definitions

Unless defined otherwise, all technical and scientific terms used herein have the same meaning as commonly understood by one of ordinary skill in the art.

The term “subject,” as used herein, refers to a human patient who is the object of treatment and/or observation.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent in certain embodiments with the meaning of “one or more,” “at least one” or “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

The terms “derived from” and “based on” when used with reference to a recombinant amino acid sequence mean that the recombinant amino acid sequence is substantially identical to the sequence of the corresponding reference amino acid sequence. For example, an Ig Fc amino acid sequence that is derived from (or based on) a wild-type Ig Fc sequence is substantially identical (e.g. shares at least 90%, at least 95%, at least 96%, at least 97%, at least 98% or at least 99% sequence identity) with the wild-type Ig Fc sequence.

The term “first-line therapy,” “first-line treatment” or “primary therapy” is a treatment regimen that is generally accepted as the initial treatment for a patient, taking into account the type and stage of a cancer. The term “second-line therapy” or “second-line treatment” is a treatment regimen that is typically administered if the first-line therapy does not provide the desired efficacy.

The term “neoadjuvant therapy” refers to treatment given as a first step to shrink a tumor before the main treatment, usually surgery, is given. Examples of neoadjuvant therapy include, but are not limited to, chemotherapy, radiation therapy, and hormone therapy. Neoadjuvant therapy may be considered as a first-line therapy.

The term “adjuvant therapy” refers to an additional cancer treatment given after the first-line treatment to lower the risk that the cancer will come back. Adjuvant therapy may include, but are not limited to, chemotherapy, radiation therapy, hormone therapy, targeted therapy (typically small molecule drugs or antibodies that target specific types of cancer cells rather than normal cells), or biological therapy (such as vaccines, cytokines, antibodies, or gene therapy, for example).

An “advanced cancer” is a cancer that has developed to the point where it cannot be safely removed or where a cure or long-term remission is highly unlikely. Cancers become advanced by growing adjacent to structures that prevent their removal or by spreading from where they started, crossing tissue lines, or to other parts of the body such as lymph nodes or other organs. Advanced cancers may be locally advanced, meaning that they have spread outside the organ of the primary site, but have not yet spread to distant sites. Advanced cancers may also be metastatic, meaning that the cancer cells have spread from the site were the cancer started (the primary site) to other more distant parts of the body (secondary sites).

A “resectable” cancer is one that can be treated by surgery. An “unresectable” cancer is one that cannot be treated by surgery, typically because the cancer has spread to the tissues surrounding the main tumor. Certain cancers may be assessed by a medical practitioner as “partially resectable” based on the degree of spread to surrounding tissues.

The term “fixed time interval” refers to the recommended schedule for administering a drug, for example, every week (QW), every two weeks (Q2W), every three weeks (Q3W) etc.

It is contemplated that any embodiment discussed herein can be implemented with respect to any method, use or composition disclosed herein.

Particular features, structures and/or characteristics described in connection with an embodiment disclosed herein may be combined with features, structures and/or characteristics described in connection with another embodiment disclosed herein in any suitable manner to provide one or more further embodiments.

It is also to be understood that the positive recitation of a feature in one embodiment, serves as a basis for excluding the feature in an alternative embodiment. For example, where a list of options is presented for a given embodiment or claim, it is to be understood that one or more option may be deleted from the list and the shortened list may form an alternative embodiment, whether or not such an alternative embodiment is specifically referred to.

### Anti-HER2 Biparatopic Antibodies

The antibodies described herein comprise an anti-HER2 biparatopic antibody that binds to two different epitopes of HER2.

The term “antibody,” as used herein, generally refers to a proteinaceous binding molecule with immunoglobulin-like functions. Typical examples of an antibody are immunoglobulins, as well as derivatives or functional fragments thereof which still retain binding specificity. Techniques for the production of antibodies are well known in the art. The term “antibody” may also include immunoglobulins of different classes (i.e. IgA, IgG, IgM, IgD and IgE) and subclasses (such as IgG₁, IgG₂, IgG₃, IgG₄, IgA₁ and IgA₂). Illustrative examples of an antibody are whole antibodies and antigen-binding fragments thereof, such as Fab fragments, F(ab′)₂, Fv fragments, single-chain Fv fragments (scFv), diabodies, domain antibodies, and combinations thereof. Domain antibodies may be single domain antibodies, single variable domain antibodies or immunoglobulin single variable domain having only one variable domain, which may be a heavy chain variable domain or a light chain variable domain, that specifically bind an antigen or epitope independently of other variable regions or domains. The term “antibody” also includes embodiments such as chimeric, single chain and humanized antibodies.

A typical whole antibody comprises at least two heavy (H) chains and two light (L) chains interconnected by disulfide bonds. Each heavy chain is comprised of a heavy chain variable region (VH) and a heavy chain constant region (CH). The heavy chain constant region comprises three domains: CH1, CH2 and CH3. The heavy chain constant domains that correspond to the different classes of immunoglobulins are known as α (IgA), δ (IgD), ε (IgE), γ (IgG) and u (IgM). Each light chain is comprised of a light chain variable region (VL) and a light chain constant region. The light chain constant region comprises just one domain: CL. Light chains are classified as either kappa or lambda. The VH and VL regions can be further subdivided into regions of hypervariability, termed Complementarity Determining Regions (CDR), interspersed with regions that are more conserved, termed framework regions (FW). Each VH and VL is composed of three CDRs and four FWs, arranged from amino-terminus to carboxy-terminus in the following order: FW1, CDR1, FW2, CDR2, FW3, CDR3, FW4. The variable regions of the heavy and light chains contain a binding domain (a paratope) that interacts with an antigen. The constant regions of the antibodies can mediate the binding of the immunoglobulin to host tissues or factors, including various cells of the immune system (such as effector cells) and C1q, which is a component of the complement system.

In certain embodiments, the anti-HER2 biparatopic antibodies described herein comprise two as antigen-binding domains, each of which binds to a different epitope of HER2. The terms “antigen-binding polypeptide construct” and “antigen-binding domain,” as used interchangeably herein, refer to an immunoglobulin-based construct, for example, an antibody fragment. In some embodiments, the antigen-binding polypeptide constructs comprised by the anti-HER2 biparatopic antibody are antibody fragments.

In certain embodiments, the antigen-binding polypeptide constructs comprised by the anti-HER2 biparatopic antibodies may each independently be a Fab fragment, a Fab′ fragment, an scFv or an sdAb. In some embodiments, the antigen-binding polypeptide constructs comprised by the anti-HER2 biparatopic antibody may each independently be a Fab fragment or an scFv. In some embodiments, one antigen-binding polypeptide construct comprised by the anti-HER2 biparatopic antibody may be a Fab fragment and the other antigen-binding polypeptide construct may be an scFv.

In certain embodiments, at least one of the antigen-binding polypeptide constructs comprised by the anti-HER2 biparatopic antibody may be a Fab fragment or a Fab′ fragment. A “Fab fragment” contains the constant domain of the light chain (CL) and the first constant domain of the heavy chain (CH1) along with the variable domains of the light and heavy chains (VL and VH, respectively). Fab′ fragments differ from Fab fragments by the addition of a few amino acid residues at the C-terminus of the heavy chain CH1 domain, including one or more cysteines from the antibody hinge region. A Fab fragment may also be a single-chain Fab molecule, i.e. a Fab molecule in which the Fab light chain and the Fab heavy chain are connected by a peptide linker to form a single peptide chain. For example, the C-terminus of the Fab light chain may be connected to the N-terminus of the Fab heavy chain in the single-chain Fab molecule.

In certain embodiments, at least one of the antigen-binding polypeptide constructs comprised by the anti-HER2 biparatopic antibody may be a single-chain Fv (scFv). An “scFv” includes a heavy chain variable domain (VH) and a light chain variable domain (VL) of an antibody in a single polypeptide chain. The scFv may optionally further comprise a polypeptide linker between the VH and VL domains which enables the scFv to form a desired structure for antigen binding. For example, an scFv may include a VL connected from its C-terminus to the N-terminus of a VH by a polypeptide linker. Alternately, an scFv may comprise a VH connected through its C-terminus to the N-terminus of a VL by a polypeptide chain or linker (see review in Pluckthun in The Pharmacology of Monoclonal Antibodies, vol. 113, Rosenburg and Moore eds., Springer-Verlag, New York, pp. 269-315 (1994)).

The anti-HER2 biparatopic antibodies described herein may have various formats. The minimal components of the anti-HER2 biparatopic antibody are a first antigen-binding polypeptide construct that binds to a first HER2 epitope and a second antigen-binding polypeptide construct that binds to a second HER2 epitope, with the first and second HER2 epitopes being different. An antibody that comprises two antigen-binding polypeptide constructs that bind to different HER2 epitopes may be considered to be a bivalent, biparatopic antibody. Antibodies that comprise one or more additional antigen-binding polypeptide constructs, each of which binds to either the first or second HER2 epitope, are also biparatopic, but are considered to be trivalent or tetravalent, for example. In certain embodiments, the anti-HER2 biparatopic antibody is a bivalent, anti-HER2 biparatopic antibody.

In certain embodiments, the anti-HER2 biparatopic antibody comprises a scaffold to which first and second antigen-binding polypeptide constructs are operably linked. The term “operably linked,” as used herein, means that the components described are in a relationship permitting them to function in their intended manner. Suitable scaffolds are described below. In some embodiments, the anti-HER2 biparatopic antibody comprises two antigen-binding polypeptide constructs operably linked to a scaffold, and at least one of the antigen-binding polypeptide constructs is an scFv. In some embodiments, the anti-HER2 biparatopic antibody comprises two antigen-binding polypeptide constructs operably linked to a scaffold, and at least one of the antigen-binding polypeptide constructs is a Fab. In some embodiments, the anti-HER2 biparatopic antibody comprises two antigen-binding polypeptide constructs operably linked to a scaffold, where one of the antigen-binding polypeptide constructs is an scFv and the other antigen-binding polypeptide construct is a Fab.

Examples of suitable scaffolds include, but are not limited to, immunoglobulin Fc regions, albumin, albumin analogs and derivatives, heterodimerizing peptides (such as leucine zippers, heterodimer-forming “zipper” peptides derived from Jun and Fos, IgG CH1 and CL domains or barnase-barstar toxins), cytokines, chemokines or growth factors. Other examples include antibodies based on the DOCK-AND-LOCK™ (DNL™) technology developed by IBC Pharmaceuticals, Inc. and Immunomedics, Inc. (see, for example, Chang, et al., 2007, Clin Cancer Res., 13: 5586s-5591s).

In certain embodiments, the anti-HER2 biparatopic antibody comprises a scaffold that is based on an immunoglobulin Fc region, an albumin or an albumin analogue or derivative (such as those described in International Patent Application Publication No. WO 2012/116453 or WO 2014/012082). In some embodiments, the anti-HER2 biparatopic antibody comprises a protein scaffold that is based on an immunoglobulin (Ig) Fc region. In some embodiments, the anti-HER2 biparatopic antibody comprises a protein scaffold that is based on an IgG Fc region.

The terms “Fc region,” “Fc” or “Fc domain” as used herein refer to a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, MD (1991).

Ig Fc regions are typically dimeric and composed of two Fc polypeptides. An “Fc polypeptide” of a dimeric Fc refers to one of the two polypeptides forming the dimeric Fc domain, i.e. a polypeptide comprising one or more C-terminal constant regions of an immunoglobulin heavy chain that is capable of stable self-association. The terms “first Fc polypeptide” and “second Fc polypeptide” may be used interchangeably to describe the Fc polypeptides comprised by a dimeric Fc region, provided that the Fc region comprises one first Fc polypeptide and one second Fc polypeptide.

An Fc region comprises a CH3 domain or both a CH3 and a CH2 domain. For example, an Fc polypeptide of a dimeric IgG Fc region comprises an IgG CH2 and an IgG CH3 constant domain sequence. The CH3 domain comprises two CH3 sequences, one from each of the two Fc polypeptides of the dimeric Fc region. The CH2 domain comprises two CH2 sequences, one from each of the two Fc polypeptides of the dimeric Fc region.

In some embodiments, the anti-HER2 biparatopic antibody may comprise a scaffold that is based on an IgG Fc region. In some embodiments, the anti-HER2 biparatopic antibody may comprise a scaffold that is based on a human Fc region. In some embodiments, the anti-HER2 biparatopic antibody may comprise a scaffold based on a human IgG Fc region, for example a human IgG1 Fc region.

In certain embodiments, the anti-HER2 biparatopic antibody may comprise a scaffold based on an IgG Fc region, which is a heterodimeric Fc region, comprising a first Fc polypeptide and a second Fc polypeptide, each comprising a CH3 sequence, and optionally a CH2 sequence.

In some embodiments, the anti-HER2 biparatopic antibody may comprise a scaffold based on an Fc region which comprises first and second Fc polypeptides, and the first antigen-binding polypeptide construct is operably linked to the first Fc polypeptide and the second antigen-binding polypeptide construct is operably linked to the second Fc polypeptide.

In some embodiments, the anti-HER2 biparatopic antibody may comprise a scaffold based on an Fc region which comprises first and second Fc polypeptides, in which the first antigen-binding polypeptide construct is operably linked to the first Fc polypeptide and the second antigen-binding polypeptide construct is operably linked to the second Fc polypeptide, and in which the first and second antigen-binding polypeptide constructs are independently a Fab fragment or an scFv.

In some embodiments, the anti-HER2 biparatopic antibody may comprise a scaffold based on an Fc region which comprises two CH3 sequences, at least one of which comprises one or more amino acid modifications. In some embodiments, the anti-HER2 biparatopic antibody comprises a heterodimeric Fc region comprising a modified CH3 domain, wherein the modified CH3 domain is an asymmetrically modified CH3 domain. Generally, the first Fc polypeptide of the heterodimeric Fc comprises a first CH3 sequence and the second Fc polypeptide comprises a second CH3 sequence.

As used herein, “asymmetric amino acid modification” refers to a modification where an amino acid at a specific position on a first CH3 sequence is different to the amino acid on a second CH3 sequence at the same position. For CH3 sequences comprising asymmetric amino acid modifications, the first and second CH3 sequence will typically preferentially pair to form a heterodimer, rather than a homodimer. These asymmetric amino acid modifications can be a result of modification of only one of the two amino acids at the same respective amino acid position on each sequence, or different modifications of both amino acids on each sequence at the same respective position on each of the first and second CH3 sequences. Each of the first and second CH3 sequence of a heterodimeric Fc may comprise one or more than one asymmetric amino acid modification.

In certain embodiments, the anti-HER2 biparatopic antibody may comprise a scaffold based on a modified Fc region as described in International Patent Application Publication No. WO 2012/058768 or WO 2013/063702.

Table 1 provides the amino acid sequence of the human IgG1 Fe sequence (SEQ ID NO: 1), corresponding to amino acids 231 to 447 of the full-length human IgG1 heavy chain. The CH3 sequence comprises amino acids 341-447 of the full-length human IgG1 heavy chain.

In certain embodiments, the anti-HER2 biparatopic antibody may comprise a heterodimeric Fc scaffold comprising a modified CH3 domain that comprises asymmetric amino acid modifications that promote formation of a heterodimeric Fc rather than a homodimeric Fc. In some embodiments, the anti-HER2 biparatopic antibody may comprise a heterodimeric Fc scaffold which includes modifications as described below at one or more of the following positions: L351, F405, Y407, T366, K392, T394, T350, S400 and/or N390, using EU numbering.

In certain embodiments, the anti-HER2 biparatopic antibody may comprise a heterodimeric Fc comprising a modified CH3 domain having a first polypeptide sequence that comprises amino acid modifications at positions F405 and Y407, and optionally further comprises an amino acid modification at position L351, and a second polypeptide sequence that comprises amino acid modifications at positions T366 and T394, and optionally further comprises an amino acid modification at position K392. In some embodiments, a first polypeptide sequence of the modified CH3 domain may comprise amino acid modifications at positions F405 and Y407, and optionally further comprises an amino acid modification at position L351, and a second polypeptide sequence of the modified CH3 domain comprises amino acid modifications at positions T366 and T394, and optionally further comprises an amino acid modification at position K392, and the amino acid modification at position F405 is F405A, F4051, F405M, F405S, F405T or F405V; the amino acid modification at position Y407 is Y4071 or Y407V; the amino acid modification at position T366 is T366I, T366L or T366M; the amino acid modification at position T394 is T394W; the amino acid modification at position L351 is L351Y, and the amino acid modification at position K392 is K392F, K392L or K392M. In some embodiments, the amino acid modification at position F405 is F405A, F405S, F405T or F405V.

In some embodiments, the anti-HER2 biparatopic antibody may comprise a heterodimeric Fc comprising a modified CH3 domain having a first Fc polypeptide sequence comprising amino acid modifications at positions F405 and Y407, and optionally further comprises an amino acid modification at position L351, and a second Fc polypeptide sequence comprising amino acid modifications at positions T366 and T394, and optionally further comprises an amino acid modification at position K392, and the amino acid modification at position F405 is F405A, F4051, F405M, F405S, F405T or F405V; the amino acid modification at position Y407 is Y4071 or Y407V; the amino acid modification at position T366 is T366I, T366L or T366M; the amino acid modification at position T394 is T394W; the amino acid modification at position L351 is L351Y, and the amino acid modification at position K392 is K392F, K392L or K392M, and one or both of the first and second Fc polypeptide sequences further comprises the amino acid modification T350V. In some embodiments, the amino acid modification at position F405 is F405A, F405S, F405T or F405V.

In certain embodiments, the anti-HER2 biparatopic antibody may comprise a heterodimeric Fc comprising a modified CH3 domain as described above, in which the first Fc polypeptide sequence comprises amino acid modifications at positions F405 and Y407, and optionally further comprises an amino acid modification at position L351, and the second Fc polypeptide sequence comprises amino acid modifications at positions T366 and T394, and optionally further comprises an amino acid modification at position K392, and in which the first Fc polypeptide sequence further comprises an amino acid modification at one or both of positions S400 or Q347 and/or the second Fc polypeptide sequence further comprises an amino acid modification at one or both of positions K360 or N390, where the amino acid modification at position S400 is S400E, S400D, S400R or S400K; the amino acid modification at position Q347 is Q347R, Q347E or Q347K; the amino acid modification at position K360 is K360D or K360E, and the amino acid modification at position N390 is N390R, N390K or N390D. In some embodiments, the amino acid modification at position F405 is F405A, F405S, F405T or F405V.

In certain embodiments, the anti-HER2 biparatopic antibody may comprise a heterodimeric Fc scaffold having a modified CH3 domain comprising the modifications of any one of Variant 1, Variant 2, Variant 3, Variant 4 or Variant 5, as shown in Table 1.

TABLE 1
IgG1 Fc sequences

| | |
|---|---|
| Human IgG1 | APELLGGPSVFLFPPKPKDTLMISRTPEVTCVVVDVS |
| Fc sequence | HEDPEVKFNWYVDGVEVHNAKTKPREEQYNSTYRVVS |
| 231-447 | VLTVLHQDWLNGKEYKCKVSNKALPAPIEKTISKAKG |
| (EU- | QPREPQVYTLPPSRDELTKNQVSLTCLVKGFYPSDIA |
| numbering) | VEWESNGQPENNYKTTPPVLDSDGSFFLYSKLTVDKS |
| | RWQQGNVFSCSVMHEALHNHYTQKSLSLSPGK |
| | (SEQ ID NO: 1) |

| Variant | | |
|---|---|---|
| IgG1 Fc | | |
| sequence | Chain | Mutations |
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

In certain embodiments, the anti-HER2 biparatopic antibody may comprise a heterodimeric Fc scaffold having a modified CH3 domain with a first CH3 sequence comprising one or more amino acid modifications selected from L351Y, F405A, and Y407V, and the second CH3 sequence comprising the amino acid modifications T366L or T366I; K392L or K392M, and T394W, and one or both of the first and second CH3 sequences may optionally further comprise the amino acid modification T350V.

The two antigen-binding polypeptide constructs comprised by the anti-HER2 biparatopic antibody each bind to a different epitope of HER2, that is, a first antigen-binding polypeptide construct binds to a first HER2 epitope and a second antigen-binding polypeptide construct binds to a second HER2 epitope. In the context of the present disclosure, each of the antigen-binding polypeptide constructs specifically binds to its target epitope.

“Specifically binds” or “specific binding” mean that the binding is selective for the antigen and can be discriminated from unwanted or non-specific interactions. The ability of an antigen-binding polypeptide construct to bind to a specific epitope can be measured, for example, through an enzyme-linked immunosorbent assay (ELISA), surface plasmon resonance (SPR) techniques (analyzed on a BIAcore instrument) (Liljeblad et al, Glyco J 17, 323-329 (2000)) or traditional binding assays (Heeley, Endocr Res 28, 217-229 (2002)). In some embodiments, the antigen-binding polypeptide construct is considered to specifically bind to its target epitope when the extent of binding of the antigen-binding polypeptide construct to an unrelated protein is less than about 10% of the binding of the antigen-binding polypeptide construct to its target epitope as measured, for example, by SPR.

“HER2” (also known as ErbB2) refers to human HER2 protein described, for example, in Semba et al., PNAS (USA), 82:6497-6501 (1985) and Yamamoto et al., Nature, 319:230-234 (1986) (GenBank accession number X03363). The terms “erbB2” and “neu” refer to the gene encoding human HER2 protein. The terms p185 or p185neu may also be used to refer to the protein product of the neu gene.

HER2 comprises an extracellular domain, which typically binds a HER ligand, a lipophilic transmembrane domain, a conserved intracellular tyrosine kinase domain and a carboxyl-terminal signaling domain harboring several tyrosine residues which can be phosphorylated. The extracellular (ecto) domain of HER2 comprises four domains, Domains I-IV. The sequence of HER2 is provided in Table 2 (SEQ ID NO:2). The Extracellular Domain (ECD) boundaries are: Domain I-approximately amino acids 1-165; Domain II-approximately amino acids 166-322; Domain III-approximately amino acids 323-488, and Domain IV-approximately amino acids 489-607.

TABLE 2
Amino Acid Sequence of Human HER2 (SEQ ID NO: 2)

| | |
|---|---|
| 1 | TQVCTGTDMKLRLPASPETHLDMLRHLYQGCQVVQGNLELTYLPTNASLSFLQDIQEVQG |
| 61 | YVLIAHNQVRQVPLQRLRIVRGTQLFEDNYALAVLDNGDPLNNTTPVTGASPGGLRELQL |
| 121 | RSLTEILKGGVLIQRNPQLCYQDTILWKDIFHKNNQLALTLIDTNRSRACHPCSPMCKGS |
| 181 | RCWGESSEDCQSLTRTVCAGGCARCKGPLPTDCCHEQCAAGCTGPKHSDCLACLHENHSG |
| 241 | ICELHCPALVTYNTDTFESMPNPEGRYTFGASCVTACPYNYLSTDVGSCTLVCPLHNQEV |
| 301 | TAEDGTQRCEKCSKPCARVCYGLGMEHLREVRAVTSANIQEFAGCKKIFGSLAFLPESFD |
| 361 | GDPASNTAPLQPEQLQVFETLEEITGYLYISAWPDSLPDLSVFQNLQVIRGRILHNGAYS |
| 421 | LTLQGLGISWLGLRSLRELGSGLALIHHNTHLCFVHTVPWDQLFRNPHQALLHTANRPED |
| 481 | ECVGEGLACHQLCARGHCWGPGPTQCVNCSQFLRGQECVEECRVLQGLPREYVNARHCLP |
| 541 | CHPECQPQNGSVTCFGPEADQCVACAHYKDPPFCVARCPSGVKPDLSYMPIWKFPDEEGA |
| 601 | CQPCPIN |

“Epitope 2C4” is the region in the extracellular domain of HER2 to which the antibody 2C4 binds and comprises residues from Domain II in the extracellular domain of HER2 (also referred to as ECD2). 2C4 and Pertuzumab bind to the extracellular domain of HER2 at the junction of Domains I, II and III (Franklin et al. Cancer Cell 5:317-328 (2004)).

“Epitope 4D5” is the region in the extracellular domain of HER2 to which the antibody 4D5 (ATCC CRL 10463) and trastuzumab bind. This epitope is close to the transmembrane domain of HER2, and within Domain IV of HER2 (also referred to as ECD4).

In general, the anti-HER2 biparatopic antibody of the present disclosure will bind to epitopes within the extracellular domains of HER2. In some embodiments, the first and second HER2 epitopes bound by the first and second antigen-binding polypeptide constructs of the anti-HER2 biparatopic antibody are non-overlapping epitopes. In some embodiments, the first and second HER2 epitopes bound by the first and second antigen-binding polypeptide constructs of the anti-HER2 biparatopic antibody are on different extracellular domains of HER2. In some embodiments, the first antigen-binding polypeptide construct of the anti-HER2 biparatopic antibody binds to a first HER2 epitope on a first domain of HER2, and the second antigen-binding polypeptide construct binds to a second HER2 epitope on a second domain of HER2. In some embodiments, the first domain of HER2 is ECD2 and the second domain of HER2 is ECD4.

In certain embodiments, one of the antigen-binding polypeptide constructs comprised by the anti-HER2 biparatopic antibody competes with trastuzumab for binding to HER2. In certainembodiments, one of the antigen-binding polypeptide constructs comprised by the anti-HER2 biparatopic antibody competes with pertuzumab for binding to HER2. In certain embodiments, one of the antigen-binding polypeptide constructs comprised by the anti-HER2 biparatopic antibody competes with trastuzumab for binding to HER2, and the other antigen-binding polypeptide construct competes with pertuzumab for binding to HER2.

In certain embodiments, one of the antigen-binding polypeptide constructs comprised by the anti-HER2 biparatopic antibody is in a Fab or scFv format and competes with trastuzumab for binding to HER2, and the other antigen-binding polypeptide construct is in a Fab or scFv format and competes with pertuzumab for binding to HER2. In certainembodiments, one of the antigen-binding polypeptide constructs comprised by the anti-HER2 biparatopic antibody is in a Fab format and competes with trastuzumab for binding to HER2, and the other antigen-binding polypeptide construct is in an scFv format and competes with Pertuzumab for binding to HER2.

In some embodiments, one of the antigen-binding polypeptide constructs comprised by the anti-HER2 biparatopic antibody binds to the same epitope on HER2 as trastuzumab. In some embodiments, one of the antigen-binding polypeptide constructs comprised by the anti-HER2 biparatopic antibody binds to the same epitope on HER2 as pertuzumab. In some embodiments, one of the antigen-binding polypeptide constructs comprised by the anti-HER2 biparatopic antibody binds to the same epitope on HER2 as trastuzumab, and the other antigen-binding polypeptide construct binds to the same epitope on HER2 as pertuzumab.

In some embodiments, one of the antigen-binding polypeptide constructs comprised by the anti-HER2 biparatopic antibody comprises the CDR sequences of trastuzumab or a variant thereof comprising one or more mutations known to increase HER2 binding, and the other antigen-binding polypeptide construct comprises the CDRs of pertuzumab or a variant thereof comprising one or more mutations known to increase HER2 binding. Literature mutations known to enhance HER2 binding by trastuzumab or pertuzumab include those listed in Tables 3 and 4 below (HC=heavy chain; LC=light chain). Combinations of these mutations are also contemplated.

TABLE 3
Trastuzumab Mutations that Increase Binding to HER2

| Mutation | Reported Improvement |
|---|---|
| HC: D102W (HC: D98W) | 3.2× |
| HC: D102Y | 3.1× |
| HC: D102K | 2.3× |
| HC: D102T | 2.2× |
| HC: N55K | 2.0× |
| HC: N55T | 1.9× |
| LC: H91F | 2.1× |
| LC: D28R | 1.9× |

TABLE 4
Pertuzumab Mutations that Increase Binding to HER2

| Mutation | Reported Improvement |
|---|---|
| LC: I31A | 1.9× |
| LC: Y96A | 2.1× |
| LC: Y96F | 2.5× |
| HC: T30A | 2.1× |
| HC: G56A | 8.3× |
| HC: F63V | 1.9× |

In certain embodiments, the anti-HER2 biparatopic antibody is one of the biparatopic antibodies described in U.S. Patent Application Publication No. 2016/0289335. In some embodiments, the anti-HER2 biparatopic antibody is one of v5019, v5020, v7091, v10000, v6902, v6903 or v6717 (see Tables 5, 6 and 7, and Sequence Tables). In some embodiments, one of the antigen-binding polypeptide constructs of the anti-HER2 biparatopic antibody comprises a VH sequence and a VL sequence from the ECD2-binding arm of one of v5019, v5020, v7091, v10000, v6902, v6903 or v6717. In some embodiments, one of the antigen-binding polypeptide constructs of the anti-HER2 biparatopic antibody comprises a VH sequence and a VL sequence from the ECD2-binding arm of one of v5019, v5020, v7091, v10000, v6902, v6903 or v6717, and the other antigen-binding polypeptide construct comprises a VH sequence and a VL sequence from the ECD4-binding arm of one of v5019, v5020, v7091, v10000, v6902, v6903 or v6717. In some embodiments, one of the antigen-binding polypeptide constructs of the anti-HER2 biparatopic antibody comprises a VH sequence and a VL sequence from the ECD2-binding arm of v10000, and the other antigen-binding polypeptide construct comprises a VH sequence and a VL sequence from the ECD4-binding arm of v10000.

In certain embodiments, one of the antigen-binding polypeptide constructs of the anti-HER2 biparatopic antibody comprises the CDR sequences from the ECD2-binding arm of one of v5019, v5020, v7091, v10000, v6902, v6903 or v6717. In some embodiments, one of the antigen-binding polypeptide constructs of the anti-HER2 biparatopic antibody comprises the CDR sequences from the ECD2-binding arm of one of v5019, v5020, v7091, v10000, v6902, v6903 or v6717, and the other antigen-binding polypeptide construct comprises the CDR sequences from the ECD4-binding arm of one of v5019, v5020, v7091, v10000, v6902, v6903 or v6717. In some embodiments, one of the antigen-binding polypeptide constructs of the anti-HER2 biparatopic antibody comprises the CDR sequences from the ECD2-binding arm of v10000, and the other antigen-binding polypeptide construct comprises the CDR sequences from the ECD4-binding arm of v10000.

TABLE 5
Exemplary Anti-HER2 Biparatopic Antibodies

| Variant | | Chain A | Chain B |
|---|---|---|---|
| 5019 | Domain | ECD2 | ECD4 |
| | containing target | | |
| | epitope | | |
| | Format | Fab | scFv |
| | Antibody name | Pertuzumab | Trastuzumab |
| | CH3 sequence | T350V_L351Y_F405A_Y407V | T366I_N390R_K392M_T394W |
| | substitutions^{§} | | |
| 5020 | Domain | ECD4 | ECD2 |
| | containing target | | |
| | epitope | | |
| | Format | scFv | Fab |
| | Antibody name | Trastuzumab | Pertuzumab |
| | CH3 sequence | L351Y_S400E_F405A_Y407V | T350V_T366L_K392L_T394W |
| | substitutions | | |
| 7091 | Domain | ECD2 | ECD4 |
| | containing target | | |
| | epitope | | |
| | Format | Fab | scFv |
| | Antibody name | Pertuzumab | Trastuzumab |
| | CH3 sequence | T350V_L351Y_F405A_Y407V | T350V_T366L_K392L_T394W |
| | substitutions | | |
| 10000 | Domain | ECD2 | ECD4 |
| | containing target | | |
| | epitope | | |
| | Format | Fab | scFv |
| | Antibody name | Pertuzumab | Trastuzumab |
| | Fab sequence | HC: T30A_A49G_L69F | |
| | substitutions* | LC: Y96A | |
| | CH3 sequence | T350V_L351Y_F405A_Y407V | T350V_T366L_K392L_T394W |
| | substitutions | | |
| 6902 | Domain | ECD4 | ECD2 |
| | containing target | | |
| | epitope | | |
| | Format | Fab | Fab |
| | Antibody name | Trastuzumab | Pertuzumab |
| | Fab sequence | HC: L143E_K145T | HC: D146G_Q179K |
| | substitutions | LC: Q124R | LC: Q124E_Q160E_T180E |
| | CH3 sequence | T350V_L351Y_F405A_Y407V | T350V_T366L_K392L_T394W |
| | substitutions | | |
| 6903 | Domain | ECD4 | ECD2 |
| | containing target | | |
| | epitope | | |
| | Format | Fab | Fab |
| | Fab sequence | HC: L143E_K145T | HC: D146G_Q179K |
| | substitutions | LC: Q124R_Q1160K_T178R | LC: Q124E_Q160E_T180E |
| | Antibody name | Trastuzumab | Pertuzumab |
| | CH3 sequence | T350V_L351Y_F405A_Y407V | T350V_T366L_K392L_T394W |
| | substitutions | | |
| 6717 | Domain | ECD2 | ECD4 |
| | containing target | | |
| | epitope | | |
| | Format | scFv | scFv |
| | Antibody name | Pertuzumab | Trastuzumab |
| | CH3 sequence | T350V_L351Y_F405A_Y407V | T366I_N390R_K392M_T394W |
| | substitutions | | |

*Fab or variable domain numbering according to Kabat (Kabat et al., Sequences of proteins of immunological interest, 5^{th} Edition, US Department of Health and Human Services, NIH Publication No. 91-3242, p.647, 1991)
^{§}CH3 numbering according to EU index as in Kabat (Edelman et al., 1969, PNAS USA, 63: 78-85)

TABLE 6
CDR Sequences of the ECD2-Binding Arm of
Variants v5019, v5020, v7091, v10000,
v6902, v6903 and v6717

| | | SEQ | | SEQ |
|---|---|---|---|---|
| | | ID | | ID |
| Variant | HC CDRs | NO | LC CDRs | NO |
| 5019, 5020, | H1: GFTFTDYT | 5 | L1: QDVSIG | 10 |
| 7091, 6902, | H2: VNPNSGGS | 7 | L2: SAS | 12 |
| 6903 & 6717 | H3: ARNLGPSFYFDY | 6 | L3: QQYYIYPYT | 11 |
| 10000 | H1: GFTFADYT | 32 | L1: QDVSIG | 22 |
| | H2: VNPNSGGS | 34 | L2: SAS | 24 |
| | H3: ARNLGPSFYFDY | 33 | L3: QQYYIYPAT | 23 |

TABLE 7
CDR Sequences of the ECD4-Binding Arm of
Variants v5019, v5020, v7091, v10000,
v6902, v6903 and v6717

| | SEQ ID | | SEQ ID |
|---|---|---|---|
| HC CDRs | NO | LC CDRs | NO |
| H1: GFNIKDTY | 27, 56 | L1: QDVNTA | 53 |
| H2: IYPTNGYT | 29, 57 | L2: SAS | 54 |
| H3: SRWGGDGFYAMDY | 28, 58 | L3: QQHYTTPPT | 55 |

In certain embodiments, the anti-HER2 biparatopic antibody comprises (a) a first antigen-binding domain comprising the CDR sequences as set forth in SEQ ID NOs: 32, 34 and 33, and in SEQ ID NOs: 22, 24 and 23, and (b) a second antigen-binding domain comprising the CDR sequences as set forth in SEQ ID NOs: 53, 54 and 55, and SEQ ID NOs: 56, 57 and 58.

In certain embodiments, the anti-HER2 biparatopic antibody comprises (a) a first antigen-binding domain that is a Fab and comprises the CDR sequences as set forth in SEQ ID NOs: 32, 34 and 33, and in SEQ ID NOs: 22, 24 and 23, and (b) a second antigen-binding domain that is an scFv and comprises the CDR sequences as set forth in SEQ ID NOs: 53, 54 and 55, and SEQ ID NOs: 56, 57 and 58.

In certain embodiments, the anti-HER2 biparatopic antibody comprises (a) a first antigen-binding domain comprising a first set of CDRs comprising the CDR1 sequence as set forth in SEQ ID NO: 32, the CDR2 sequence as set forth in SEQ ID NO: 34 and the CDR3 sequence as set forth in SEQ ID NO: 33, and a second set of CDR sequences comprising the CDR1 sequence as set forth in SEQ ID NO: 22, the CDR2 sequence as set forth in SEQ ID NO: 24 and the CDR3 sequence as set forth in SEQ ID NO: 23, and (b) a second antigen-binding domain comprising a third set of CDR sequences comprising the CDR1 sequence as set forth in SEQ ID NO: 53, the CDR2 sequence as set forth in SEQ ID NO: 54 and the CDR3 sequence as set forth in SEQ ID NO: 55, and a fourth set of CDR sequences comprising the CDR1 sequence as set forth in SEQ ID NO: 56, the CDR2 sequence as set forth in SEQ ID NO: 57 and the CDR3 sequence as set forth in SEQ ID NO: 58.

In certain embodiments, the anti-HER2 biparatopic antibody comprises (a) a first heavy chain (H1) comprising the CDR sequences as set forth in SEQ ID NOs: 32, 34 and 33, (b) a second heavy chain (H2) scFv comprising the CDR sequences as set forth in SEQ ID NOs: 53, 54, 55, 56, 57 and 58, and (c) a light chain (L1) comprising the CDR sequences as set forth in SEQ ID NOS: 22, 24 and 23.

In certain embodiments, the anti-HER2 biparatopic antibody comprises (a) a first heavy chain (H1) comprising a first set of CDR sequences comprising the CDR1 sequence as set forth in SEQ ID NO: 32, the CDR2 sequence as set forth in SEQ ID NO: 34 and the CDR3 sequence as set forth in SEQ ID NO: 33, (b) a second heavy chain (H2) comprising a second set of CDR sequences comprising the CDR1 sequence as set forth in SEQ ID NO: 53, the CDR2 sequence as set forth in SEQ ID NO: 54, and the CDR3 sequence as set forth in SEQ ID NO: 55, and a third set of CDR sequences comprising the CDR1 sequence as set forth in SEQ ID NO: 56, the CDR2 sequence as set forth in SEQ ID NO: 57 and the CDR3 sequence as set forth in SEQ ID NO: 58, and a light chain (L1) comprising a fourth set of CDR sequences comprising the CDR1 sequence as set forth in SEQ ID NO: 22, the CDR2 sequence as set forth in SEQ ID NO: 24 and the CDR3 sequence as set forth in SEQ ID NO: 23.

In certain embodiments, the anti-HER2 biparatopic antibody comprises (a) a first antigen-binding domain comprising the VH sequence as set forth in SEQ ID NO: 31, and the VL sequence as set forth in SEQ ID NO: 21, and (b) a second antigen-binding domain comprising the VH sequence as set forth in SEQ ID NO: 52, and the VL sequence as set forth in SEQ ID NO: 51.

In certain embodiments, the anti-HER2 biparatopic antibody comprises (a) a first antigen-binding domain that is a Fab and comprises the VH sequence as set forth in SEQ ID NO: 31, and the VL sequence as set forth in SEQ ID NO: 21, and (b) a second antigen-binding domain that is an scFv and comprises the VH sequence as set forth in SEQ ID NO: 52, and the VL sequence as set forth in SEQ ID NO: 51.

In certain embodiments, the anti-HER2 biparatopic antibody comprises a first heavy chain (H1) comprising the VH sequence as set forth in SEQ ID NO: 31, a second heavy chain (H2) comprising the VH sequence as set forth in SEQ ID NO: 52 and the VL sequence as set forth in SEQ ID NO: 51, and a light chain (L1) comprising the VL sequence as set forth in SEQ ID NO: 21.

In certain embodiments, the anti-HER2 biparatopic antibody comprises (a) a first heavy chain (H1) comprising the sequence as set forth in SEQ ID NO: 30, a second heavy chain (H2) comprising the sequence as set forth in SEQ ID NO: 50, and a light chain (L1) comprising the sequence as set forth in SEQ ID NO: 20.

In certain embodiments, the anti-HER2 biparatopic antibody comprises (a) a first heavy chain (H1) consisting of the sequence as set forth in SEQ ID NO: 30, a second heavy chain (H2) consisting of the sequence as set forth in SEQ ID NO: 50, and a light chain (L1) consisting of the sequence as set forth in SEQ ID NO: 20.

### Preparation of Bispecific Anti-HER2 Antigen-Binding Constructs

The anti-HER2 biparatopic antibodies described herein may be produced using recombinant methods and compositions, e.g., as described in U.S. Pat. No. 4,816,567 or International Patent Publication No. WO2015/077891.

In one embodiment, isolated nucleic acid encoding a bispecific anti-HER2 biparatopic antibody described herein is provided. Such nucleic acid may encode an amino acid sequence comprising the VL and/or an amino acid sequence comprising the VH of an anti-HER2 biparatopic antibody (e.g., the light and/or heavy chains of the anti-HER2 biparatopic antibody. In a further embodiment, one or more vectors (e.g., expression vectors) comprising such nucleic acid are provided. As is known in the art, because many amino acid acids are encoded by more than one codon, multiple nucleic acids may encode a single polypeptide sequence.

In one embodiment, the nucleic acid is provided in a multicistronic vector. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the anti-HER2 biparatopic antibody and an amino acid sequence comprising the VH of the antigen-binding polypeptide construct, or (2) a first vector comprising a nucleic acid that encodes an amino acid sequence comprising the VL of the anti-HER2 biparatopic antibody and a second vector comprising a nucleic acid that encodes an amino acid sequence comprising the VH of the anti-HER2 biparatopic antibody. In one embodiment, the host cell is eukaryotic, e.g. a Chinese Hamster Ovary (CHO) cell, or human embryonic kidney (HEK) cell, or lymphoid cell (e.g., Y0, NS0, Sp20 cell). In one embodiment, a method of making an anti-HER2 biparatopic antibody is provided, wherein the method comprises culturing a host cell comprising nucleic acid encoding the anti-HER2 biparatopic antibody, as provided above, under conditions suitable for expression of the anti-HER2 biparatopic antibody, and optionally recovering the anti-HER2 biparatopic antibody from the host cell (or host cell culture medium).

For recombinant production of the anti-HER2 biparatopic antibody, nucleic acid encoding an anti-HER2 biparatopic antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the anti-HER2 biparatopic antibody).

The term “substantially purified” refers to a construct described herein, or variant thereof that may be substantially or essentially free of components that normally accompany or interact with the protein as found in its naturally occurring environment, i.e. a native cell, or host cell in the case of recombinantly produced anti-HER2 biparatopic antibody that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein. When the anti-HER2 biparatopic antibody is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the bispecific anti-HER2 antigen-binding construct is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, “substantially purified” bispecific anti-HER2 antigen-binding construct produced by the methods described herein, has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Suitable host cells for cloning or expression of anti-HER2 biparatopic antibody-encoding vectors include prokaryotic or eukaryotic cells described herein.

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

For example, anti-HER2 biparatopic antibody may be produced in bacteria, in particular when glycosylation and Fc effector function are not needed. For expression of anti-HER2 biparatopic antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the bispecific anti-HER2 antigen-binding construct may be isolated from the bacterial cell paste in a soluble fraction and can be further purified.

In addition to prokaryotes, eukaryotic microbes such as filamentous fungi or yeast are suitable cloning or expression hosts for bispecific anti-HER2 antigen-binding construct-encoding vectors, including fungi and yeast strains whose glycosylation pathways have been “humanized,” resulting in the production of an bispecific anti-HER2 antigen-binding construct with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated the anti-HER2 biparatopic antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antigen-binding constructs in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antigen-binding construct production, see, e.g., Yazaki and Wu, Methods in Molecular Biology, Vol. 248 (B.K.C. Lo, ed., Humana Press, Totowa, N.J.), pp. 255-268 (2003).

In one embodiment, the -HER2 biparatopic antibodies described herein are produced in stable mammalian cells, by a method comprising: transfecting at least one stable mammalian cell with: nucleic acid encoding the anti-HER2 biparatopic antibody, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the anti-HER2 biparatopic antibody in the expressed product.

In some embodiments the anti-HER2 biparatopic antibody is produced in stable mammalian cells wherein the expression product of the at least one stable mammalian cell comprises a larger percentage of the desired glycosylated the anti-HER2 biparatopic antibody as compared to the monomeric heavy or light chain polypeptides, or other antibodies. In some embodiments, identification of the glycosylated anti-HER2 biparatopic antibody is by one or both of liquid chromatography and mass spectrometry.

If required, the anti-HER2 biparatopic antibodies can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use for purification of the anti-HER2 biparatopic antibodies described herein. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni⁺² affinity chromatography if a His-tag is employed, or immobilized anti-flag antibody if a flag-tag is used. For general guidance in suitable purification techniques, see, e.g. incorporated entirely by reference Protein Purification: Principles and Practice, 3^{rd} Ed., Scopes, Springer-Verlag, NY, 1994, incorporated entirely by reference. The degree of purification necessary will vary depending on the use of the bispecific anti-HER2 antigen-binding constructs. In some instances no purification is necessary.

In certain embodiments the anti-HER2 biparatopic antibodies are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments the anti-HER2 biparatopic antibody described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, anti-HER2 biparatopic antibody constructs described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, the D-isomers of the common amino acids, 2,4diaminobutyric acid, alpha-amino isobutyric acid, 4aminobutyric acid, Abu, 2-amino butyric acid, γ-Abu, ε-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, N α-methyl amino acids, and amino acid analogs in general. Furthermore, the amino acid can be D (dextrorotary) or L (levorotary).

### Post-Translational Modifications:

In certain embodiments anti-HER2 biparatopic antibodies described herein are differentially modified during or after translation.

The term “modified,” as used herein refers to any changes made to a given polypeptide, such as changes to the length of the polypeptide, the amino acid sequence, chemical structure, co-translational modification, or post-translational modification of a polypeptide. The form “(modified)” term means that the polypeptides being discussed are optionally modified, that is, the polypeptides of the bispecific anti-HER2 antigen-binding construct can be modified or unmodified.

The term “post-translationally modified” refers to any modification of a natural or non-natural amino acid that occurs to such an amino acid after it has been incorporated into a polypeptide chain. The term encompasses, by way of example only, co-translational in vivo modifications, co-translational in vitro modifications (such as in a cell-free translation system), post-translational in vivo modifications, and post-translational in vitro modifications.

In some embodiments, the modification is at least one of: glycosylation, acetylation, phosphorylation, amidation, derivatization by known protecting/blocking groups, proteolytic cleavage and linkage to an antibody molecule or anti-HER2 biparatopic antibody or other cellular ligand. In some embodiments, the anti-HER2 biparatopic antibody is chemically modified by known techniques, including but not limited, to specific chemical cleavage by cyanogen bromide, trypsin, chymotrypsin, papain, V8 protease, NaBH₄; acetylation, formylation, oxidation, reduction; and metabolic synthesis in the presence of tunicamycin.

Additional post-translational modifications of anti-HER2 biparatopic antibodies include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The bispecific anti-HER2 antigen-binding constructs described herein are modified with a detectable label, such as an enzymatic, fluorescent, isotopic or affinity label to allow for detection and isolation of the protein. In certain embodiments, examples of suitable enzyme labels include horseradish peroxidase, alkaline phosphatase, beta-galactosidase, or acetylcholinesterase; examples of suitable prosthetic group complexes include streptavidin biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin; and examples of suitable radioactive material include iodine, carbon, sulfur, tritium, indium, technetium, thallium, gallium, palladium, molybdenum, xenon, fluorine.

In specific embodiments, anti-HER2 biparatopic antibodies described herein are attached to macrocyclic chelators that associate with radiometal ions.

In some embodiments, the anti-HER2 biparatopic antibodies described herein are modified by either natural processes, such as post-translational processing, or by chemical modification techniques which are well known in the art. In certain embodiments, the same type of modification may be present in the same or varying degrees at several sites in a given polypeptide. In certain embodiments, polypeptides from anti-HER2 biparatopic antibodies described herein are branched, for example, as a result of ubiquitination, and in some embodiments are cyclic, with or without branching. Cyclic, branched, and branched cyclic polypeptides are a result from posttranslation natural processes or made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York, pgs. 1-12 (1983); Seifter et al., Meth. Enzymol. 182:626-646 (1990); Rattan et al., Ann. N.Y. Acad. Sci. 663:48-62 (1992))

### Pharmaceutical Compositions

For therapeutic use, the anti-HER2 biparatopic antibodies may be provided in the form of compositions comprising the antibodyand a pharmaceutically acceptable carrier or diluent. The compositions may be prepared by known procedures using well-known and readily available ingredients.

Pharmaceutical compositions may be formulated for administration to a subject by, for example, oral (including, for example, buccal or sublingual), topical, parenteral, rectal or vaginal routes, or by inhalation or spray. The term “parenteral” as used herein includes subcutaneous injection, and intradermal, intra-articular, intravenous, intramuscular, intravascular, intrasternal, intrathecal injection or infusion. The pharmaceutical composition will typically be formulated in a format suitable for administration to the subject by the selected route, for example, as a syrup, elixir, tablet, troche, lozenge, hard or soft capsule, pill, suppository, oily or aqueous suspension, dispersible powder or granule, emulsion, injectable or solution. Pharmaceutical compositions may be provided as unit dosage formulations.

In certain embodiments, the pharmaceutical compositions comprising the anti-HER2 biparatopic antibodies are formulated for parenteral administration in injectable form, for example as lyophilized formulations or aqueous solutions.

Pharmaceutically acceptable carriers are generally nontoxic to recipients at the dosages and concentrations employed. Examples of such carriers include, but are not limited to, buffers such as phosphate, citrate, and other organic acids; antioxidants such as ascorbic acid and methionine; preservatives such as octadecyldimethylbenzyl ammonium chloride, hexamethonium chloride, benzalkonium chloride, benzethonium chloride, phenol, butyl alcohol, benzyl alcohol, alkyl parabens (such as methyl or propyl paraben), catechol, resorcinol, cyclohexanol, 3-pentanol and m-cresol; low molecular weight (less than about 10 residues) polypeptides; proteins such as serum albumin or gelatin; hydrophilic polymers such as polyvinylpyrrolidone; amino acids such as glycine, glutamine, asparagine, histidine, arginine or lysine; monosaccharides, disaccharides, and other carbohydrates such as glucose, mannose or dextrins; chelating agents such as EDTA; sugars such as sucrose, mannitol, trehalose or sorbitol; salt-forming counter-ions such as sodium; metal complexes such as Zn-protein complexes, and non-ionic surfactants such as polyethylene glycol (PEG).

In certain embodiments, the compositions comprising the anti-HER2 biparatopic antibodies may be in the form of a sterile injectable aqueous or oleaginous solution or suspension. Such suspensions may be formulated using suitable dispersing or wetting agents and/or suspending agent that are known in the art. The sterile injectable solution or suspension may comprise the anti-HER2 biparatopic antibody in a non-toxic parentally acceptable diluent or carrier. Acceptable diluents and carriers that may be employed include, for example, 1,3-butanediol, water, Ringer's solution, isotonic sodium chloride solution or dextrose. In addition, sterile, fixed oils may be employed as a carrier. For this purpose, various bland fixed oils may be employed, including synthetic mono- or diglycerides. In addition, fatty acids such as oleic acid find use in the preparation of injectables. Adjuvants such as local anaesthetics, preservatives and/or buffering agents may also be included in the injectable solution or suspension.

In certain embodiments, the composition comprising the anti-HER2 biparatopic antibodies may be formulated for intravenous administration to humans. Typically, compositions for intravenous administration are solutions in sterile isotonic aqueous solution, for example, containing sodium chloride or dextrose. Where necessary, the composition may also include a solubilizing agent and/or a local anaesthetic such as lignocaine to case pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water, saline or dextrose. Where the composition is administered by injection, an ampoule of sterile water for injection or saline can be provided so that the ingredients may be mixed prior to administration.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Methods of Use

Certain aspects of the present disclosure relate to methods of treating a HER2-expressing cancer in a subject by administering an effective amount of an anti-HER2 biparatopic antibody as described herein.

HER2-expressing cancers are typically solid tumors. Examples of HER2-expressing solid tumors include, but are not limited to, breast cancer, endometrial cancer, ovarian cancer, cervical cancer, lung cancer, gastric cancer, esophageal cancer, colorectal cancer, anal cancer, urothelial cancer, pancreatic cancer, salivary gland cancer and brain cancer. HER2-expressing breast cancer include estrogen receptor negative (ER-) and/or progesterone receptor negative (PR-) breast cancers and triple negative (ER-, PR-, low HER2) breast cancers. HER2-expressing lung cancers include non-small cell lung cancer (NSCLC) and small cell lung cancer.

In certain embodiments, the methods described herein are for the treatment of HER2-expressing solid tumor. In some embodiments, the methods described herein are for the treatment of a HER2-expressing breast cancer, gastroesophageal adenocarcinoma (GEA), esophageal cancer, endometrial cancer, ovarian cancer, cervical cancer, non-small cell lung cancer (NSCLC), anal cancer or colorectal cancer (CRC).

In certain embodiments, the methods described herein are for the treatment of a HER2-expressing cancer that is metastatic or locally advanced. In some embodiments, the methods described herein are for the treatment of a HER2-expressing cancer that has metastasized to the brain. In certain embodiments, the methods described herein are for first line treatment of a HER2-expressing cancer. In certain embodiments, the methods described herein are for second line treatment of a HER2-expressing cancer.

As is known in the art, HER2-expressing cancers may be characterized by the level of HER2 they express (i.e. by “HER2 status”). HER2 status can be assessed, for example, by immunohistochemistry (IHC), fluorescent in situ hybridization (FISH) and chromogenic in situ hybridization (CISH) or DNA in situ hybridization (ISH, for example DNAscope™). A number of commercial kits are available for assessing HER2 status in patients. Examples of FDA-approved commercial kits available for HER2 detection using IHC include HercepTest™ (Dako Denmark A/S); PATHWAY (Ventana Medical Systems, Inc.); InSite™HER2/NEU kit (Biogenex Laboratories, Inc.) and Bond Oracle HER2 IHC System (Leica Biosystems.

IHC identifies HER2 protein expression on the cell membrane. For example, paraffin-embedded tissue sections from a tumor biopsy may be subjected to the IHC assay and accorded a HER2 staining intensity criteria as follows:
Score 0: no staining observed or membrane staining is observed in less than 10% of tumor cells; typically <20,000 receptors/cell.
Score 1+: a faint/barely perceptible membrane staining is detected in more than 10% of the tumor cells. The cells are only stained in part of their membrane. Typically about 100,000 receptors/cell.
Score 2+: a weak to moderate complete membrane staining is observed in more than 10% of the tumor cells; typically about 500,000 receptors/cell.
Score 3+: a moderate to strong complete membrane staining is observed in more than 10% of the tumor cells; typically about 2,000,000 receptors/cell.

The anti-HER2 biparatopic antibodies described herein may be useful in methods of treating cancers that express HER2 at various levels. In certain embodiments, the methods of treating HER2-expressing cancers according to the present disclosure comprise administering an anti-HER2 biparatopic antibody as described herein to a subject having a cancer that expresses high levels of HER2 (HER2-high) defined as IHC 3+. In some embodiments, the methods of treating HER2-expressing cancers according to the present disclosure comprise administering an anti-HER2 biparatopic antibody as described herein to a subject having a cancer that expresses high levels of HER2 (high-HER2) defined as IHC 2+, IHC 2+/3+ or IHC 3+. In some embodiments, the methods of treating HER2-expressing cancers according to the present disclosure comprise administering an anti-HER2 biparatopic antibody as described herein to a subject having a cancer that expresses low levels of HER2 (low-HER2) defined as IHC 1+ or IHC 1+/2+. In certain embodiments, the cancer has an amplified HER2 gene that is detectable using FISH assay or an ISH assay. In certain embodiments the cancer is HER2 3+ as determined by IHC without HER2 gene amplification as detected by a FISH assay or an ISH assay. In certain embodiments the cancer is HER2 2+ as determined by IHC and has HER2 gene amplification as determined by a FISH assay. In certain embodiments, the cancer is HER2 2/3+ as determined by IHC and has HER2 gene amplification as determined by a FISH assay or an ISH assay.

In certain embodiments, the methods described herein are for the first line treatment of a subject having a HER2-expressing cancer. In certain embodiments, the methods described herein are for the second line treatment of a subject having a HER2-expressing cancer.

In certain embodiments, the methods described herein are for the treatment of a subject having a HER2-expressing cancer that is resistant or becoming resistant to other standard-of-care therapies. In some embodiments, the methods described herein are for the treatment of a subject having a HER2-expressing cancer who is unresponsive to one or more current therapies, such as trastuzumab (Herceptin®), pertuzumab (Perjeta®), T-DM1 (Kadcyla® or trastuzumab emtansine), Enhertu™ (fam-trastuzumab deruxtecan-nxki), or taxanes (such as such as paclitaxel, docetaxel, cabazitaxel, and the like). In some embodiments, the methods described herein are for the treatment of a subject having a HER2-expressing cancer that is resistant to trastuzumab. In some embodiments, the methods described herein are for the treatment of a subject having metastatic cancer that has progressed on previous anti-HER2 therapy. In some embodiments, the methods described herein are for the treatment of a subject who has previously undergone treatment with one or more of trastuzumab, pertuzumab, T-DM1 and Enhertu™ (fam-trastuzumab deruxtecan-nxki).

In certain aspects, the method of treating a subject having a HER2-expressing cancer comprises administering to the subject an effective amount of an anti-HER2 biparatopic antibody, wherein the effective amount is administered to the subject at a fixed dose at a fixed time interval.

In certain embodiments of the method, the fixed dose is selected from a low fixed dose for a subject whose weight is less than a dose cut-off weight, and a higher fixed dose for a subject whose weight is more than a dose cut-off weight.

In certain embodiments of the method, the low fixed dose is about 600 mg and the high fixed dose is about 800 mg, the dose cut-off weight is 70 kg and the fixed time internal is weekly (QW).

In certain embodiments of the method, the low fixed dose is about 800 mg and the high fixed dose is about 1200 mg, the dose cut-off weight is 70 kg and the fixed time internal is weekly (QW).

In certain embodiments of the method, the low fixed dose is about 800 mg and the high fixed dose is about 1000 mg, the dose cut-off weight is 70 kg and the fixed time internal is weekly (QW).

In certain embodiments of the method, the low fixed dose is about 1800 mg and the high fixed dose is about 2200 mg, the dose cut-off weight is 70 kg and the fixed time internal is every 2 weeks (Q2W).

In certain embodiments of the method, the low fixed dose is about 1200 mg and the high fixed dose is about 1600 mg, the dose cut-off weight is 70 kg and the fixed time internal is every 2 weeks (Q2W).

In certain embodiments of the method, the low fixed dose is about 1200 mg and the high fixed dose is about 1800 mg, the dose cut-off weight is 70 kg and the fixed time internal is every 3 weeks (Q3W).

In certain embodiments of the method, the low fixed dose is about 1800 mg and the high fixed dose is about 2400 mg, the dose cut-off weight is 70 kg and the fixed time internal is every 3 weeks (Q3W).

In certain embodiments of the method, the anti-HER2 biparatopic antibody administered to the subject comprises (a) a first antigen-binding domain comprising the CDR sequences as set forth in SEQ ID NOs: 32, 34 and 33, and in SEQ ID NOs: 22, 24 and 23, and (b) a second antigen-binding domain comprising the CDR sequences as set forth in SEQ ID NOs: 53, 54 and 55, and SEQ ID NOs: 56, 57 and 58.

In some embodiments of the method, the first antigen-binding domain of the anti-HER2 biparatopic antibody administered to the subject is a Fab and the second antigen-binding domain of the anti-HER2 biparatopic antibody administered to the subject is an scFv.

In certain embodiments of the method the anti-HER2 biparatopic antibody administered to the subject comprises a heavy chain H1 comprising the sequence set forth in SEQ ID NO. 30, a heavy chain H2 comprising the sequence set forth in SEQ ID NO: 50 and a light chain L1, comprising the sequence set forth in SEQ ID NO. 20.

In certain embodiments of the method, the HER2-expressing cancer is a solid tumor.

In certain embodiments of the method, the HER2-expressing cancer the HER2-expressing cancer is breast cancer, biliary tract cancer, gastroesophageal adenocarcinoma (GEA), esophageal cancer, gastroesophageal cancer (GEJ), gastric cancer, endometrial cancer, ovarian cancer, cervical cancer, non-small cell lung cancer (NSCLC), anal cancer or colorectal cancer (CRC).

In certain embodiments of the method, the HER2-expressing cancer the HER2-expressing cancer is gastroesophageal adenocarcinoma (GEA).

In certain embodiments of the method, the subject has received prior treatment with one or more of trastuzumab, pertuzumab, T-DM1 or Enhertu™ (fam-trastuzumab deruxtecan-nxki).

In certain embodiments of the method, the subject has not received prior treatment with an anti-HER2 targeted therapy.

In certain embodiments of the method, the subject has not received prior systemic treatment with a chemotherapeutic agent for the HER2 expressing cancer being treated.

In certain embodiments of the method, HER2-expressing cancer is metastatic.

In certain embodiments of the method, HER2-expressing cancer is is locally advanced.

In certain embodiments of the method, the HER2-expressing cancer is HER2 3+, HER2 2+/3+ or HER2 2+ or HER2 1+ as measured by immunohistochemistry (IHC) and gene amplified as measured by fluorescence in situ hybridization (FISH).

In certain embodiments of the method, the HER2-expressing cancer is HER2 3+, HER2 2+/3+ or HER2 2+ or HER2 1+ as measured by immunohistochemistry (IHC) without HER2 gene amplification as measured by fluorescence in situ hyrbridization (FISH).

In certain embodiments of the method, the HER2-expressing cancer is HER2 3+ as measured by IHC, or HER2 2+ and gene amplified as measured by FISH.

Another aspect of the present disclosure is an anti-HER2 biparatopic antibody for use in the treatment of a HER2-expressing cancer, wherein an effective dose of the antibody is a tiered fixed dose comprising a low fixed dose for a subject whose weight is less than a dose cut-off weight, and a high fixed dose for a subject whose weight is more than a dose cut-off weight. In certain embodiments, the low fixed dose is about 800 mg and the high fixed dose is about 1200 mg, the dose cut-off weight is 70 kg and the fixed time internal is weekly (QW). In some embodiments the low fixed dose is about 1200 mg and the high fixed dose is about 1600 mg, the dose cut-off weight is 70 kg and the fixed time internal is every 2 weeks (Q2W). In some embodiments the low fixed dose is about 1800 mg and the high fixed dose is about 2400 mg, the dose cut-off weight is 70 kg and the fixed time internal is every 3 weeks (Q3W). In some embodiments, the antibody is v10000.

In another aspect, the present disclosure relates to a method of treating a subject having a HER2-expressing cancer comprising administering to the subject an effective amount of an anti-HER2 biparatopic antibody, the effective amount comprising a fixed dose of 1800 mg to a subject weighing less than 70 kg, or a fixed dose of 2400 mg to a subject weighing 70 kg or more, wherein the dose is administered every 3 weeks (Q3W), wherein the subject has been diagnosed with breast cancer, gastroesophageal adenocarcinoma (GEA), esophageal cancer, gastric cancer, endometrial cancer, ovarian cancer, cervical cancer, non-small cell lung cancer (NSCLC), anal cancer or colorectal cancer (CRC).

In another aspect, the present disclosure relates to a method of treating a subject having a HER2-expressing cancer comprising administering to the subject an effective amount of an anti-HER2 biparatopic antibody, the effective amount comprising a fixed dose of 1200 mg to a subject weighing less than 70 kg, or a fixed dose of 1600 mg to a subject weighing 70 kg or more, wherein the dose is administered every 2 weeks (Q2W).

In another aspect, the present disclosure relates to a method of treating a subject having a HER2-expressing cancer comprising administering to the subject an effective amount of an anti-HER2 biparatopic antibody, the effective amount comprising a fixed dose of 1200 mg to a subject weighing less than 70 kg, or a fixed dose of 1600 mg to a subject weighing 70 kg or more, wherein the dose is administered every 2 weeks (Q2W), wherein the subject has been diagnosed with biliary tract cancer.

It is to be understood that the dosing regimens described herein are the recommended doses for administration of the anti-HER2 biparatopic antibodies, but that the doses administered may be reduced if a subject experiences adverse side effects with treatment. Similarly, the fixed interval between dosing may be altered slightly for convenience.

### Combination Therapy

Various chemotherapy regimens may be used in conjunction with the anti-HER2 biparatopic antibodies in the context of a 2-tiered dosing regimen. In certain embodiments the chemotherapy regimen is administered in accordance with the approved dose and dosing schedule for the chemotherapeutic agents used. In some embodiments, the doses of the chemotherapeutic agents may be reduced after the first cycle of treatment for reasons of tolerability.

In certain embodiments, the chemotherapy regimen comprises one or more of paclitaxel, capecitabine, mFOLFOX6 (fluorouracil+leucovorin+oxaliplatin), fulvestrant+palbociclib, capecitabine+oxaliplatin (CAPOX; also called XELOX), vinorelbine, and cisplatin+fluorouracil (FP).

CAPOX (also known as XELOX is a multi-agent chemotherapy regimen consisting of capecitabine and oxaliplatin. XELOX has been established as an efficacious cytotoxic regimen for the treatment of various GEAs, including colorectal and colon cancers and advanced biliary system adenocarcinoma CAPOX is also used as an adjuvant therapy.

In certain embodiments, the anti-HER2 biparatopic antibody is administered in combination with CAPOX using the following dosages and schedules:
(a) anti-HER2 biparatopic antibody administered at a dosage of 1800 mg (subjects<70 kg) or 2400 mg (subjects≥70 kg) IV Q3W; Day 1 of each 21-day cycle;
(b) CAPOX administered as follows: capecitabine 1,000 mg/m² PO bid (total daily dose of 2000 mg/m²) on Days 1-14 of each 21-day cycle plus oxaliplatin 130 mg/m² IV Q3W dosing on Day 1 of each 21-day cycle.

FP is a multi-agent chemotherapy regimen consisting of 5-FU and cisplatin. FP has been established as an efficacious cytotoxic regimen for the treatment of gastric cancer and is also used as a neoadjuvant/adjuvant therapy. FP has been evaluated in combination with trastuzumab HER2-positive advanced gastric or GEJ cancer in a Phase 3, open-label, randomized, controlled trial and is currently considered the standard of care first-line chemotherapy in combination with trastuzumab in HER2 overexpressed gastroesophageal cancers. In certain embodiments, the anti-HER2 biparatopic antibody is administered in conjunction with FP using the following dosages and schedules:
(a) anti-HER2 biparatopic antibody administered at a dosage of 1800 mg (subjects<70 kg) or 2400 mg (subjects≥70 kg) IV Q3W; Day 1 of each 21-day cycle;
(b) FP administered as follows: 5-FU 800 mg/m²/day continuous IV infusion Days 1-5 of each 21-day cycle plus cisplatin 80 mg/m² IV Q3W on Day 1 of each 21-day cycle.

mFOLFOX6 is a multi-agent chemotherapy regimen consisting of oxaliplatin, leucovorin, and 5-FU. mFOLFOX has been established as an efficacious cytotoxic regimen with a manageable safety profile in various cancers. In certain embodiments, the anti-HER2 biparatopic antibody is administered in conjunction with mFOLFOX6 using the following dosages and schedules:
(a) anti-HER2 biparatopic antibody administered at a dosage of 1200 mg (subjects<70 kg) or 16000 mg (subjects≥70 kg) IV Q2W; Days 1 and 15 of each 28-day cycle;
(b) mFOLFOX6 administered as follows: 400 mg/m² IV bolus, leucovorin 400 mg/m² IV, and oxaliplatin 85 mg/m² IV Q2W on Days 1 and 15 of each 28-day cycle; 5-FU 1200 mg/m² IV continuous infusion on each day for a total of 2400 mg/m² over approximately 46 to 48 hours Q2W on Days 1 and 2 and Days 15 and 16 of each 28-day cycle.

### Other Combination Therapies

Palbociclib is an inhibitor of CDK4 and CDK6. Cyclin DI and CDK4/6 are downstream of signaling pathways which lead to cellular proliferation. In vitro, palbociclib reduced cellular proliferation of estrogen receptor (ER)-positive breast cancer cell lines by blocking progression of the cell from G1 into S phase of the cell cycle. Palbociclib is approved for the treatment of hormone receptor (HR)-positive, HER2-negative advanced or metastatic breast cancer in combination with fulvestrant in patients with disease progression following endocrine therapy. The recommended dose of palbociclib is a 125 mg capsule taken orally (PO) with food once daily (QD) for 21 consecutive days followed by 7 days off treatment during a 28-day treatment cycle (IBRANCE®).

Fulvestrant is an estrogen receptor (ER) antagonist that binds to the ER in a competitive manner with affinity comparable to that of estradiol and downregulates the ER protein in human breast cancer cells. Fulvestrant is approved for the treatment of HR-positive, HER2-negative advanced or metastatic breast cancer in combination with palbociclib in patients with disease progression after endocrine therapy. The recommended dose of fulvestrant is 500 mg to be administered IM into the buttocks (gluteal area) slowly (1 to 2 minutes per injection) as two 5-mL injections, one in each buttock, on Days 1, 15, and 29 and once monthly thereafter (FASLODEX®).

In certain embodiments, the biparatopic anti-HER2 antibody is administered in combination with palbociclib and fulvestrant according to the dosing regimens described above.

Certain aspects of the present disclosure relate to methods of treating a HER2-expressing cancer in a subject by administering an effective amount of an anti-HER2 biparatopic antibody as described herein, in combination with a checkpoint inhibitor. In certain embodiments, the checkpoint inhibitor is a PD-1 inhibitor, for example, an anti-PD-1 antibody. Examples of anti-PD-1 antibodies include, but are not limited to, pembrolizumab (Keytruda®), nivolumab (Opdivo®), cemiplimab (Libtayo®), JTX-4014 (Jounce Therapeutics), spartalizumab (PDR001) (Novartis), camrelizumab (SHR1210) (Jiangsu HengRui Medicine Co., Ltd.), sinitilimab (Innovent, Eli-Lilly), tislelizumab (BGB-A317) (Beigene), toripalimab (JS 001) (Junshi Biosciences), dostarlimab (GlaxoSmith Kline), INCMGA00012 (MGA012) (Incyte, MacroGenics), AMP-224 (AstraZeneca/MedImmune and GlaxoSmithKline) and AMP-514 (MEDI0680) (AstraZeneca).

Certain embodiments relate to methods of treating a HER2-expressing cancer in a subject by administering an effective amount of an anti-HER2 biparatopic antibody as described herein, in combination with an anti-PD-1 antibody. Certain embodiments relate to methods of treating a HER2-expressing cancer in a subject by administering an effective amount of an anti-HER2 biparatopic antibody as described herein, in combination with tisleizumab. In certain embodiments, tisleizumab is administered at a flat dose of 200 mg (independent of subject weight) Q3W. In certain embodiments a HER2-expressing cancer in a subject is treated by administering an effective amount of an anti-HER2 biparatopic antibody as described herein, in combination with pembrolizumab. In certain embodiments pembrolizumab is administered at a flat dose of 200 mg Q3W or 400 mg Q6W.

In certain embodiments, the anti-HER2 biparatopic antibody to be used in combination with an anti-PD-1 antibody administered at a dose of 1800 mg (subject weight less than 70 kg) or 2400 mg (subject weight greater to or equal to 70 kg) and is administered on Day 1 of a 21 day cycle, and tisleizumab is administered at a fixed dose of 200 mg (independent of subject weight) Q3W.

Certain embodiments relate to methods of treating a HER2 expressing cancer in a subject by administering an effective amount of an anti-HER2 biparatopic antibody as described herein, in combination with an anti-CD47 antibody or a CD47 blocker. CD47 is a widely expressed cell surface protein that functions as a marker of self. CD47 provides a “don't eat me” anti-phagocytic signal that distinguishes viable/healthy cells from apoptotic/abnormal cells. SIRPα is the CD47 receptor on macrophages. CD47 binding to this receptor inhibits phagocytosis of healthy cells, while cells displaying low levels of CD47 are susceptible to macrophage-mediated destruction. Tumor cells overexpress CD47 to evade the macrophage component of immune surveillance, and abundant CD47 expression has been observed in a wide variety of hematologic and solid tumors. In certain embodiments, an anti-HER2 biparatopic antibody is administered in conjunction with evorpacept (ALX148), a CD47-blocking myeloid checkpoint inhibitor, with evorpacept being administered at a weight-based dose of 10 mg/kg body weight QW, or 30 mg/kg body weight Q2W. In certain embodiments, the anti-HER2 biparatopic antibody v10000 is administered using a 2-tiered flat dosing regimen wherein the low fixed dose is 1800 mg and the high fixed dose is 2400 mg, and the dose cut-off weight is 70 kg (Q3W).

Certain embodiments relate to methods of treating a HER2 expressing cancer in a subject by administering an effective amount of an anti-HER2 biparatopic antibody as described herein, in combination with another anti-HER2 agent that has a different mechanism of action. In certain embodiments, the anti-HER2 biparatopic antibody is administered in combination with Tucatinib, (TUKYSA®) an oral medicine that is a tyrosine kinase inhibitor of the HER2 protein. In certain embodiments Tucatinib is administered orally twice daily at 300 mg/dose.

### Pharmaceutical Kits

Certain embodiments provide for pharmaceutical kits comprising an anti-HER2 biparatopic antibodies as described herein.

The kit typically will comprise one or more containers and a label and/or package insert on or associated with the container. The label or package insert contains instructions customarily included in commercial packages of therapeutic products, providing information about the indications, usage, dosage, administration, contraindications and/or warnings concerning the use of such therapeutic products. For example, the label or package insert may specify that the anti-HER2 biparatopic antibody is for administration Q3W at a fixed dose of about 1800 mg (for subjects weighing less than 70 kg) or a fixed dose of 2400 mg for subjects weighing 70 kg or more; or for administration Q2W at a fixed dose of 1200 mg (for subjects weighing less than 70 kg) or a fixed dose of 1600 mg (for subjects weighing 70 kg or more).

The kit may comprise a container comprising 1800 mg of v10000. The kit may comprise a container comprising 2400 mg of v10000. The kit may contain six containers each comprising 300 mg of v10000, and a package insert specifying that the six vials are to be used to treat a subject weighing less than 70 kg. The kit may comprise eight containers each comprising 300 mg of v10000, and a package insert specifying that the eight vials are to be used to treat a subject weighing 70 kg or more. The kit may comprise three containers each comprising 600 mg of v10000, and a package insert specifying that the three containers are to be used to treat a subject weighing less than 70 kg. The kit may comprise four containers each comprising 600 mg of v10000, and a package insert specifying that the six containers are to be used to treat a subject weighing 70 kg or more.

The label or package insert for the pharmaceutical kit may indicate that the anti-HER2 biparatopic antibody is to be used to treat HER2-expressing cancers which may include breast cancer, biliary tract cancer, gastroesophageal adenocarcinoma (GEA), gastroesophageal esophageal junction cancer (GEJ), gastric cancer, endometrial cancer, ovarian cancer, cervical cancer, non-small cell lung cancer (NSCLC), anal cancer or colorectal cancer (CRC).

The label or package insert for the pharmaceutical kit may indicate that the HER2-expressing cancer being treated is metastatic or locally advanced.

The label or package insert for the pharmaceutical kit may indicate that the anti-HER2 biparatopic antibody is suitable for administration in combination with an anti-PD-1 antibody.

The label or package insert for the pharmaceutical kit may indicate that the anti-HER2 biparatopic antibody is suitable for administration in combination with mFOLFOX6 (5-FU and leucovorin plus oxaliplatin), CAPOX (capecitabine plus oxaliplatin) or FP (fluorouracil [5-FU] plus cisplatin).

The label or package insert for the pharmaceutical kit may indicate that the anti-HER2 biparatopic antibody is suitable for administration in combination with another anti-HER2 agent, optionally Tucatinib.

The label or package insert may further include a notice in the form prescribed by a governmental agency regulating the manufacture, use or sale of pharmaceuticals or biological products, which notice reflects approval by the agency of manufacture, for use or sale for human or animal administration. The label or package insert also indicates that the anti-HER2 biparatopic antibody is for use to treat a HER2-expressing cancer. The container holds a composition comprising the anti-HER2 biparatopic antibody and may in some embodiments have a sterile access port (for example, the container may be an intravenous solution bag or a vial having a stopper that may be pierced by a hypodermic injection needle).

In addition to the container containing the composition comprising the anti-HER2 biparatopic antibody, the kit may comprise one or more additional containers comprising other components of the kit. For example, a pharmaceutically-acceptable buffer, such as bacteriostatic water for injection (BWFI), phosphate-buffered saline, Ringer's solution or dextrose solution; other buffers or diluents.

Suitable containers include, for example, bottles, vials, syringes, intravenous solution bags, and the like. The containers may be formed from a variety of materials such as glass or plastic. If appropriate, one or more components of the kit may be lyophilized or provided in a dry form, such as a powder or granules, and the kit can additionally contain a suitable solvent for reconstitution of the lyophilized or dried component(s).

The kit may further include other materials desirable from a commercial or user standpoint, such as filters, needles, and syringes.

The following Examples are provided for illustrative purposes and are not intended to limit the scope of the invention in any way.

### EXAMPLES

### Example 1: Description and Preparation of Variant 10000 (V10000)

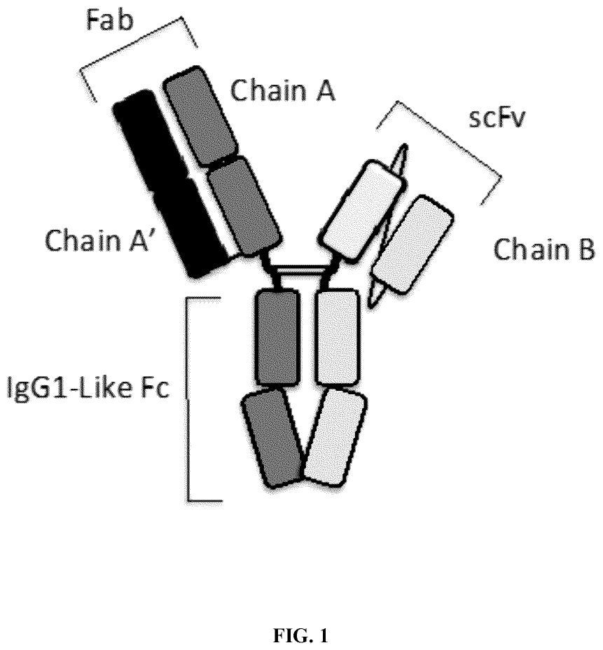

v10000 is a humanized bispecific antibody that recognizes 2 non-overlapping epitopes of the ECD of the human HER2 antigen. The IgG1-like Fc region of v10000 contains complementary mutations in each CH3 domain that impart preferential pairing to generate a heterodimeric molecule and correspondingly disfavor formation of homodimers. FIG. 1 depicts a representation of the format of v10000 where heavy chain A and light chain A′ form the ECD2 binding portion of the antibody and heavy chain B comprises the scFv that forms the ECD4 binding portion of the antibody. Variant 10000 comprises a heavy chain H1 (corresponding to heavy chain A in FIG. 1) comprising the sequence set forth in SEQ ID NO:30, a heavy chain H2 (corresponding to heavy chain B in FIG. 1) comprising the sequence set forth in SEQ ID NO:50, and a light chain L1 (corresponding to light chain A′) comprising the sequence set forth in SEQ ID NO: 20. Methods of preparing v10000 are described in detail in International Patent Publication No. WO 2015/077891.

v10000 was manufactured according to the relevant regulatory requirements for human trials and formulated at 15 mg/mL in biocompatible aqueous buffer, for IV infusion at ambient temperature. v10000 was supplied in a vial containing 300 mg v10000 in 20 mL buffer. Vials of v10000 were shipped frozen and stored at −20° C. (+/−5° C.) until ready for use. Vials were thawed at ambient temperature prior to use. Thawed solutions in vials were stored for up to 24 hours at ambient temperatures or up to 72 hours at refrigerated conditions (2° C. to 8° C.) and used before the labeled expiration date.

### Example 2: Pharmacokinetic Modeling Of v10000

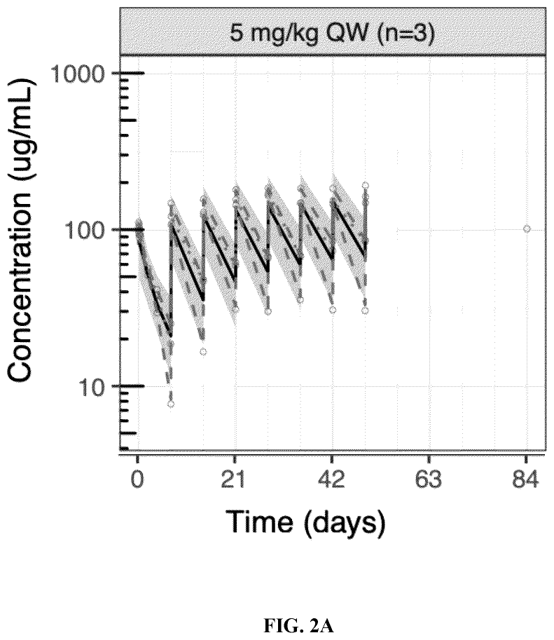

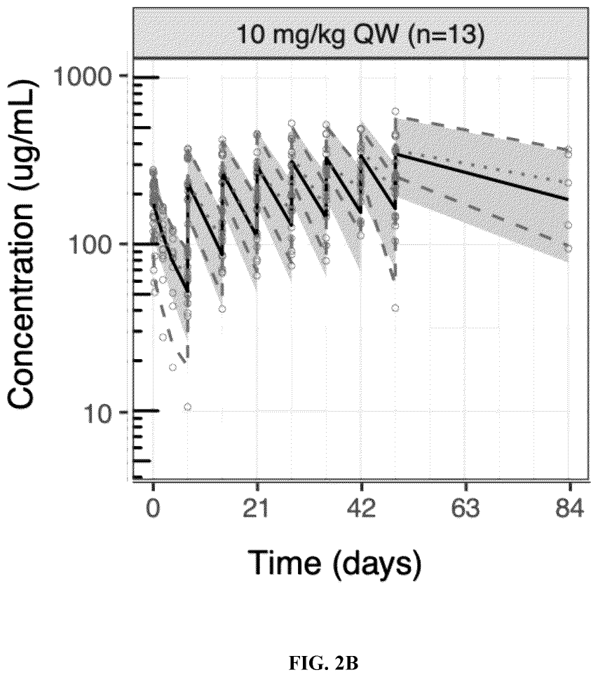

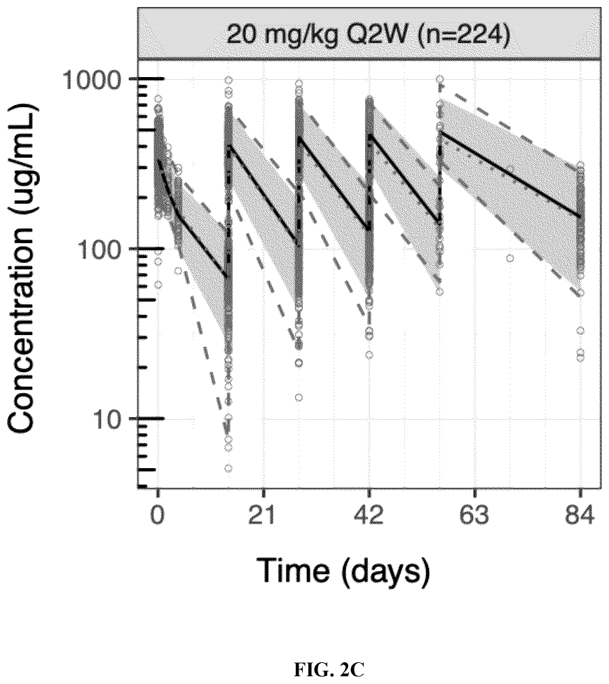

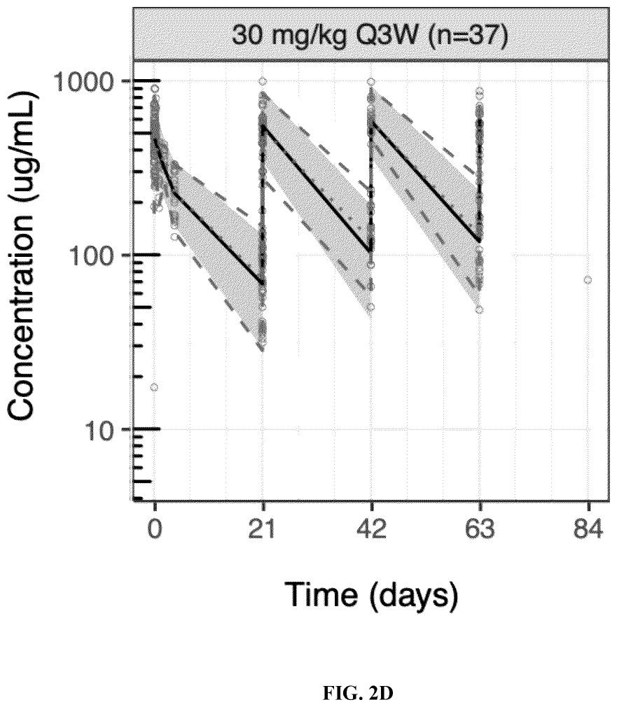

Population PK modeling was used to simulate the exposure of anti-HER2 antibody v10000 in subjects intravenously injected according to several dose regimens of the antibody (FIG. 2): (A) 10 mg/kg QW; (B) 20 mg/kg Q2Wand (C) 30 mg/kg Q3W.

To improve caregiver convenience and reduce wastage of drug product, flat (or fixed) dosing of v10000 was evaluated by simulation using a population PK model. The influence of body weight on exposure was estimated using a power model on body weight covariate terms for volume of central compartment and clearance.

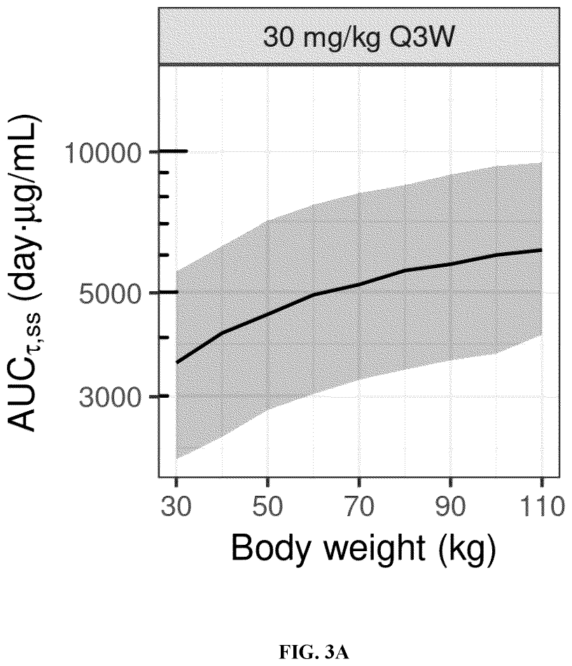

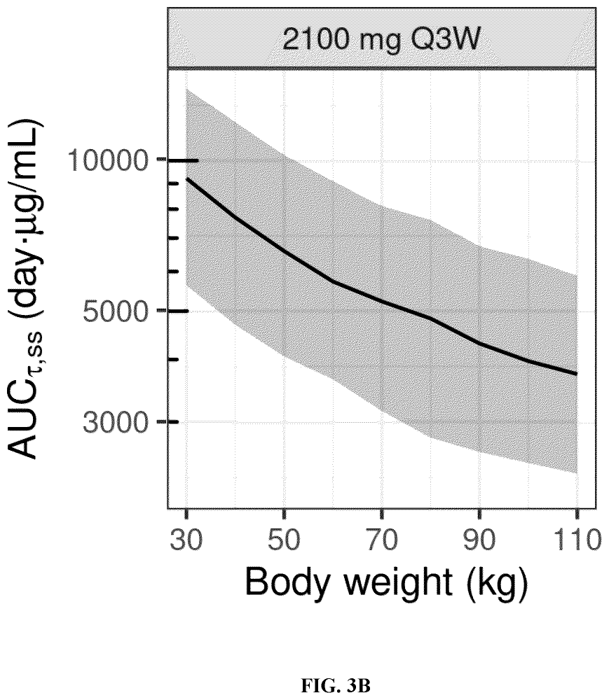

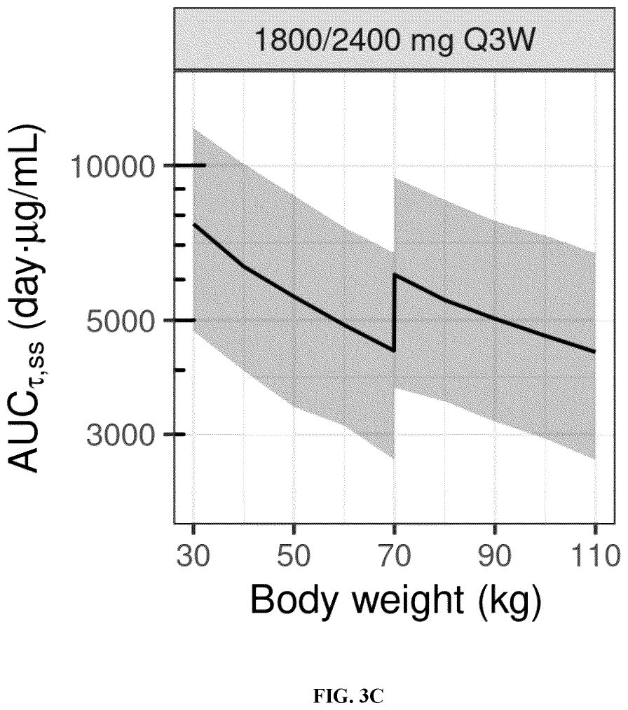

Variability of v10000 was compared through simulation of weight-based and flat dosing using the population PK model. FIG. 3 shows a comparison of model-predicted steady state trough concentration of weight-based, (A) flat (B), and two-tiered flat dosing (C) in subjects diagnosed with GEA based on Q3W administration of v10000.

Both flat (CV: 43.5%) and weight-based dosing (CV: 43.4%) resulted similar variation in steady state trough concentration. Based on the population PK model simulation, higher body weights tended to have higher exposure with the body weight-scaled dosing, while lower body weights have higher exposure with flat dosing (FIG. 3). A hybrid approach between weight-based and flat dosing utilizing a two-tiered flat dose with weight cutoff point at 70 kilograms (<70 kg, ≥70 kg) may result in more consistent exposure across body weights compared to single-tier flat and/or weight-based dosing.

The drug exposure was simulated from the population PK model by sampling from the observed covariates from 305 subjects participating in four clinical trials, and sampling from the model-fitted inter-individual variability, and fixed effects uncertainty.

### Example 3: In Vivo Phamacokinetics of v10000

The pharmacokinetic parameters of v10000 that was administered either on a weight-based dosing regimen of 30 mg/kg Q3W or on a two-tiered fixed (flat) dosing regimen of 1800 mg for clinical trial subjects weighing less than 70 kg and 2400 mg for subjects weighing 70 kg or more are shown in Table 8. It can be seen that the weight-based and flat dosing regimens resulted in similar pharmacokinetics. The pharmacokinetic parameters of v10000 that was administered either on a weight-based dosing regimen of 20 mg/kg Q2W or on a two-tiered fixed (flat) dosing regimen of 1200 mg for subjects weighing less than 70 kg and 1600 mg for subjects weighing 70 kg or more are shown in Table 8. Again, the weight-based and flat dosing regiments resulted in similar pharmacokinetics.

TABLE 8
Pharmacokinetic Parameters of v10000^{g}*

| | | | | | | AUC_{0-∞} | | |
|---|---|---|---|---|---|---|---|---|
| | | Cₘₐₓ | C_{trough} | t_{1/2} | AUC₀₋ₜ | (d*mcg/ | V_{z} | CL |
| Dosage | n | (mcg/mL) | (mcg/mL) | (d) | (d*mcg/mL) | mL) | (mL/kg) | (mL/h/kg) |
| 20 mg/kg Q2W^{a} | 24 | 430 (22) | 72 (41) | 7.2 (30) | 2365 (21) | 3195 (25) | 65 (27) | 0.26 (25) |
| 20 mg/kg Q2W^{b} | 8 | 377 (18) | 56 (30) | 7 (14) | 1897 (16) | 2492 (18) | 81 (16) | 0.33 (18) |
| 1200/1600 mg | 4 | 409 (20) | 50 (61) | 5 (34) | 2013 (18) | 2456 (26) | 57 (26) | 0.32 (28) |
| Q2W^{c} | | | | | | | | |
| 30 mg/kg Q3W^{d} | 10 | 630 (20) | 108 (16) | 11.1 (15) | 4933 (20) | 6707 (16) | 71 (23) | 0.20 (16) |
| 30 mg/kg Q3W^{e} | 11 | 418 (19) | 50 (32) | 8.7 (12) | 3348 (29) | 4018 (26) | 94 (30) | 0.31 (26) |
| 1800/2400 mg | 4 | 593 (17) | 51 (18) | 7.9 (11) | 3899 (16) | 4504 (12) | 68 (22) | 0.25 (15) |
| Q3W^{f} | | | | | | | | |

Cₘₐₓ = maximum observed concentration of drug in the serum or plasma; C_{trough} = observed concentration at the end of dosing interval; t_{1/2} = an estimate of the terminal half-life of the drug in serum or plasma calculated by dividing the natural log of 2 by the terminal elimination rate constant; AUC₀₋ₜ = AUC from time zero to time t; AUC_{0-∞} = AUC from time zero to infinity; CL = serum clearance; Vz = terminal elimination phase.
^{a}Non-GEA (Breast Cancer, Colorectal cancer, Biliary Tract Cancer, All other)
^{b}Metastatic Gastric/Gastroesophageal Junction Adenocarcinoma
^{c}Metastatic Gastric/Gastroesophageal Junction Adenocarcinoma
^{d}Metastatic Breast Cancer
^{e}Metastatic Gastric/Gastroesophageal Junction Adenocarcinoma
^{f}Metastatic Gastric/Gastroesophageal Junction Adenocarcinoma
^{g}*Values are expressed as geometric mean (coefficient of variation)

### Example 4: Anti-Tumor Effect of Subjects Dosed with V100000 Using Both a Weight-Based and a 2-Tiered Fixed Dosing Regimen

A Phase 2 clinical study of the anti-HER2 biparatopic antibody v10000 (see Example 1) as a first line treatment in patients with locally advanced (unresectable) and/or metastatic HER2-experessing gastrointestinal cancers is being conducted.

This is a multicenter, global, Phase 2, open-label, first-line, 2-part study to investigate the safety, tolerability, and anti-tumor activity of v10000, an anti-HER2 biparatopic antibody (see Example 1) plus physician's choice of combination chemotherapy. Physician's choice of combination chemotherapy includes 3 globally-recognized, multi-agent, first-line treatments:
(1) XELOX, which consists of capecitabine plus oxaliplatin
   Three variants of the XELOX and v10000 combination (XELOX-1, XELOX-2, and XELOX-3) are being tested. The variants differ in the v10000 regimen.
(2) FP, which consists of fluorouracil (5-FU) plus cisplatin

Two variants of the FP and v10000 combination (FP-1 and FP-2) are being tested. The variants differ in the v10000 regimen; and
(3) mFOLFOX6, which consists of 5-FU and leucovorin plus oxaliplatin

Two variants of the mFOLFOX6 and v10000 combination (mFOLFOX6-1 and mFOLFOX6-2) are being tested. The variants differ by the presence (mFOLFOX6-1) or absence (mFOLFOX6-2) of a 5-FU bolus on Days 1 and 15 of each 4-week treatment cycle AND by the v10000 dose (weight-based dose versus flat dose).

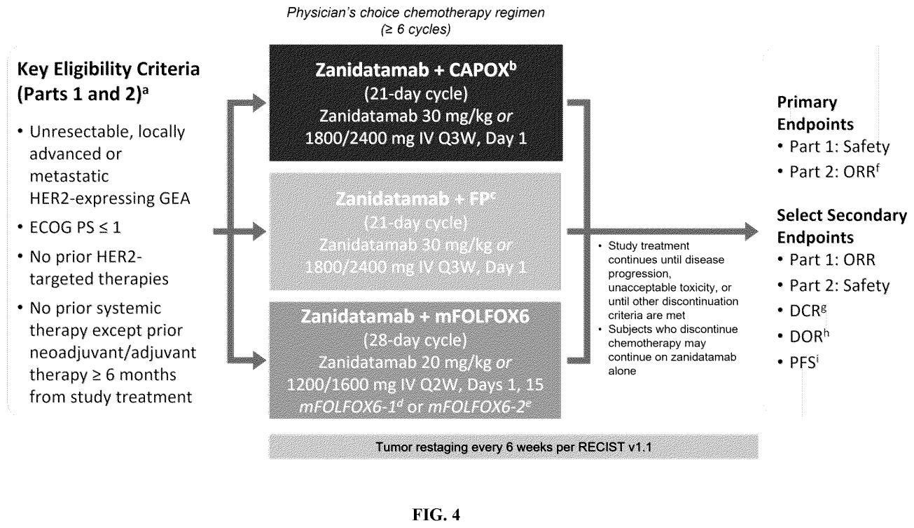

A schematic drawing of the study design is shown in FIG. 4. To be eligible for the study, subjects must have had unresectable locally advanced or metastatic GEA, GEJ or gastric cancer and have had no prior HER2 targeted therapies. Variant 10000 was administered according to either a weight-based or a two-tiered flat dosing regimen. Part 1 of the study used local or central assessment of HER2 status and allowed HER2 IHC 3+ or IHC 2+ regardless of HER2 FISH status. Part 2 included only subjects with HER2-positive cancer (IHC 3+ or IHC 2+/FISH+).

Thirty-six subjects had enrolled in the study as of the data cut off date, 9 with esophageal cancer, 14 with gastroesophageal junction cancer, and 13 with gastric cancer. The median age was 58, with a range of 27-77.

The CAPOX +z cohort received, during a 21 cycle: capecitabine 1,000 mg/m² PO BID, on Days 1-15; oxaliplatin 130 mg/m² IV Q3W, Day 1 and v10000 at either a weight-based dose of 30 mg/kg, or a 2-tiered flat dose consisting of 1800 mg for subjects under 70 kg and 2400 mg for subjects at or over 70 kg on Day 1.

The FP cohort received, during a 21-day cycle: cisplatin 80 mg/m² IV Q3W, Day 1; 5-FU 800 mg/m²/day IV, continuous Days 1-5 and v10000 at either a weight-based dose of 30 mg/kg, or a 2-tiered flat dose consisting of 1800 mg for subjects under 70 kg and 2400 mg for subjects at or over 70 kg on Day 11.

The mFOLFOX6-1cohort received, during a 28 day cycle: leucovorin 400 mg/m² IV Q2W, Days 1, 15; oxaliplatin 85 mg/m² IV Q2W, Days 1, 15; 5-FU 1200 mg/m²/day IV, continuous Days 1-2 and 15-16, and 400 mg/m² IV Q2W, Days 1, 15; and v10000 at either a weight-based dose of 20 mg/kg, or a 2-tiered flat dose consisting of 1200 mg for subjects under 70 kg and 1600 mg for subjects at or over 70 kg on Days 1, 15.

The mFOLFOX6-2 regimen is identical to the mFOLFOX6-1 regimen but omits the 5-FU 400 mg/m² IV Q2W dose on Days 1 and 15.

Part 1 of the study focused on safety and dose-limiting toxicity (DLT). The following was observed: V10000+CAPOX resulted in no DLTs in 6 subjects. V10000+FP resulted in one DLT (acute kidney injury, grade 3) in 2 subjects. V10000+mFOLFOX6-1 resulted in two DLTs (diarrhea, grade 3) in 13 subjects, and 8/13 (62%) with grade 3 diarrhea. The safety monitoring committee recommended a modified regimen (mFOLFOX6-2) that omits the 5-FU 400 mg/m² bolus on Days 1, 15. V10000+mFOLFOX6-2 resulted in one DLT (diarrhea, grade 3) in 7 subjects, and 2/7 (29%) with grade 3 diarrhea.

Part 2 of the study focused on antitumor activity of v10000 plus combination chemotherapy in subjects with HER2-positive cancer. Disease Control Rate (DCR) was defined as a best response out of Complete Response (CR), Partial Response (PR), or Stable Disease (SD). Duration of Response (DOR) was defined as time from first objective response that is subsequently confirmed to documented PD or death≤30 days of last study treatment from any cause. Progression Free Survival (PFS) was defined as the time from the first dose of study treatment to the date of documented disease progression, clinical progression, or death from any cause. 5-FU=5-fluorouracil; DCR=disease control rate; DOR=duration of response; ECOG PS=Eastern Cooperative Oncology Group performance status; FISH=fluorescence in situ hybridization; GEA=gastroesophageal adenocarcinoma; IHC=immunohistochemistry; ORR=objective response rate; PD=progressive disease; PFS=progression-free survival; RECIST v1.1=Response Evaluation Criteria in Solid Tumors, version 1.1; SD=stable disease. There were 28 efficacy-evaluable subjects in parts 1 and 2 at the data cutoff date. The top line results are shown in Table 9. The ORR was 75% and the DCR was 89%.

TABLE 9
Objective Response Rate and Disease Control Rate

| | V10000 + CAPOX^{a} | | V10000 + FP^{a} | | V10000 + mFOLFOX6^{a} | | | |
|---|---|---|---|---|---|---|---|---|
| | N = 12 | | N = 2 | | N = 14 | | Total N = 28 | |
| ^{b}cORR, % | 92 | (61.25, 99.8) | 100 | (15.8, 100) | 57 | (28.9, 82.3) | 75 | (55.1, 89.3) |
| (95% CI) | | | | | | | | |
| CR, n (%) | 0 | | 0 | | 1 | (7) | 1 | (4) |
| PR, n ( %) | 11 | (92) | 2 | (100) | 7 | (50) | 20 | (71) |
| SD, n (%) | 1 | (8) | 0 | | 3 | (21) | 4 | (14) |
| PD, n (%) | 0 | | 0 | | 3 | (21) | 3 | (11) |
| Disease | 100 | (73.5, 100) | 1100 | (15.8, 100) | 79 | (49.2, 95.3) | 89 | (71.8, 97.7) |
| Control | | | | | | | | |
| Rate, % | | | | | | | | |
| (95% CI) | | | | | | | | |

^{a}HER2-positive was defined as IHC 3+ or IHC 2+/FISH+.
^{b}cORR included a baseline scan and a confirmatory scan obtained ≥4 weeks following initial documentation of objective response; the efficacy-evaluable population was defined as all HER2-positive subjects who had ≥1 evaluable post-baseline disease assessment or discontinued study treatment due to death or clinical progression.
5-FU = 5-fluorouracil; CAPOX = capecitabine plus oxaliplatin; CR = complete response; DCR = disease control rate; FP = 5-FU and cisplatin; mFOLFOX6 = 5-FU plus oxaliplatin and leucovorin; NR = not reached; ORR = objective response rate (CR + PR); PD = progressive disease; PR = partial response; SD = stable disease.

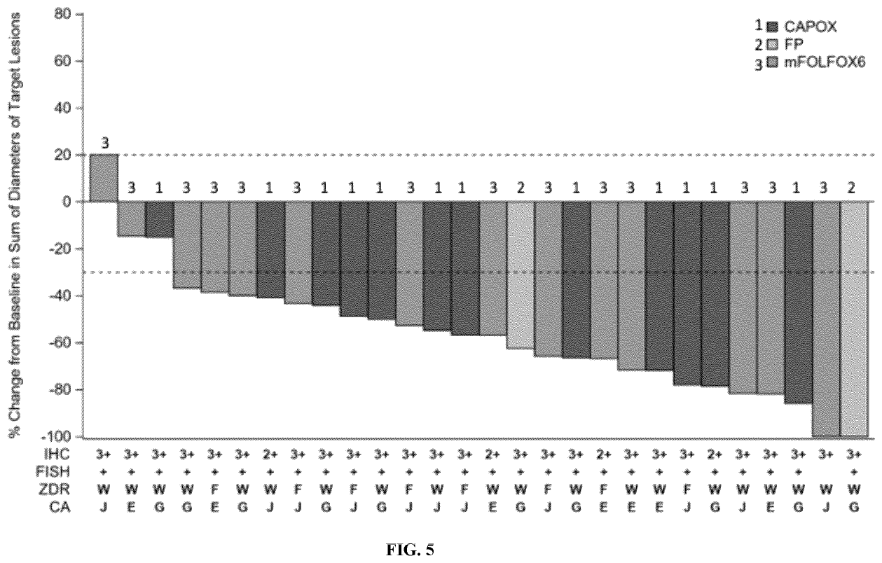

The waterfall plot in FIG. 5 shows the change in target lesion size individually for the 28 efficacy-evaluable subjects treated in the three regimens (v10000 plus CAPOX, FP or mFOLFOX). This plot shows the individuals subjects who were treated with a weight-based regimen or the 2-tiered flat dosing regimen described above. The data suggests that the 2-tiered flat dosing regimen provides comparable efficacy to the weight-based regimen. Eight out of eight (100%) of subjects treated with the 2-tiered flat dosing regimen had a target lesion size reduction of greater than 30%. Seventeen of the twenty (85%) subjects treated using the weight-based regimen had a target lesion size reduction of greater than 30%.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

### Sequence Tables

TABLE A
Clone Numbers for Variants v5019, v5020, v7091, v10000, v6903,
v6902 and v6717

| Variant | H1 clone # | H2 clone # | L1 clone # | L2 clone # |
|---|---|---|---|---|
| 5019 | 3057 | 720 | 1811 | — |
| 5020 | 719 | 3041 | — | 1811 |
| 7091 | 3057 | 5244 | 1811 | — |
| 10000 | 6586 | 5244 | 3382 | — |
| 6903 | 5065 | 3468 | 5037 | 3904 |
| 6902 | 5065 | 3468 | 5034 | 3904 |
| 6717 | 3317 | 720 | — | — |

TABLE B
Sequence for Variants v5019, v5020, v7091, v10000,
v6903, v6902 and v6717 by Clone Number

| SEQ ID | Clone | | |
|---|---|---|---|
| NO. | # | Desc | Sequence |
| 3 | 3468 | Full | EVQLVESGGGLVQPGGSLRLSCAASGFTFTDYTMDWVRQAPGKGL |
| | | | EWVADVNPNSGGSIYNQRFKGRFTLSVDRSKNTLYLQMNSLRAED |
| | | | TAVYYCARNLGPSFYFDYWGQGTLVTVSSASTKGPSVFPLAPSSK |
| | | | STSGGTAALGCLVKGYFPEPVTVSWNSGALTSGVHTFPAVLKSSG |
| | | | LYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSH |
| | | | EDPEVKFNWYVDGVEVHNAKTKPREEQYNSTYRVVSVLTVLHQDW |
| | | | LNGKEYKCKVSNKALPAPIEKTISKAKGQPREPQVYVLPPSRDEL |
| | | | TKNQVSLLCLVKGFYPSDIAVEWESNGQPENNYLTWPPVLDSDGS |
| | | | FFLYSKLTVDKSRWQQGNVFSCSVMHEALHNHYTQKSLSLSPG |
| 4 | 3468 | VH | EVQLVESGGGLVQPGGSLRLSCAASGFTFTDYTMDWVRQAPGKGL |
| | | | EWVADVNPNSGGSIYNQRFKGRFTLSVDRSKNTLYLQMNSLRAED |
| | | | TAVYYCARNLGPSFYFDYWGQGTLVTVSS |
| 5 | 3468, | H1 | GFTFTDYT |
| | 3057, | | |
| | 3041, | | |
| | 3317 | | |
| 6 | 3468, | H3 | ARNLGPSFYFDY |
| | 3057, | | |
| | 3041, | | |
| | 3317 | | |
| 7 | 3468, | H2 | VNPNSGGS |
| | 3057, | | |
| | 3041, | | |
| | 3317 | | |
| 8 | 1811 | Full | DIQMTQSPSSLSASVGDRVTITCKASQDVSIGVAWYQQKPGKAPK |
| | | | LLIYSASYRYTGVPSRFSGSGSGTDFTLTISSLQPEDFATYYCQQ |
| | | | YYIYPYTFGQGTKVEIKRTVAAPSVFIFPPSDEQLKSGTASVVCL |
| | | | LNNFYPREAKVQWKVDNALQSGNSQESVTEQDSKDSTYSLSSTLT |
| | | | LSKADYEKHKVYACEVTHQGLSSPVTKSFNRGEC |
| 9 | 1811 | VL | DIQMTQSPSSLSASVGDRVTITCKASQDVSIGVAWYQQKPGKAPK |
| | | | LLIYSASYRYTGVPSRFSGSGSGTDFTLTISSLQPEDFATYYCQQ |
| | | | YYIYPYTFGQGTKVEIK |
| 10 | 1811, | L1 | QDVSIG |
| | 3904, | | |
| | 3317 | | |
| 11 | 1811, | L3 | QQYYIYPYT |
| | 3904, | | |
| | 3317 | | |
| 12 | 1811, | L2 | SAS |
| | 3904, | | |
| | 3317 | | |
| 13 | 5034 | Full | DYKDDDDKDIQMTQSPSSLSASVGDRVTITCRASQDVNTAVAWYQ |
| | | | QKPGKAPKLLIYSASFLYSGVPSRFSGSRSGTDFTLTISSLQPED |
| | | | FATYYCQQHYTTPPTFGQGTKVEIKRTVAAPSVFIFPPSDERLKS |
| | | | GTASVVCLLNNFYPREAKVQWKVDNALQSGNSQESVTEQDSKDST |
| | | | YSLSSTLTLSKADYEKHKVYACEVTHQGLSSPVTKSFNRGEC |
| 14 | 5034 | VL | DIQMTQSPSSLSASVGDRVTITCRASQDVNTAVAWYQQKPGKAPK |
| | | | LLIYSASFLYSGVPSRFSGSRSGTDFTLTISSLQPEDFATYYCQQ |
| | | | HYTTPPTFGQGTKVEIK |
| 15 | 5037 | Full | DYKDDDDKDIQMTQSPSSLSASVGDRVTITCRASQDVNTAVAWYQ |
| | | | QKPGKAPKLLIYSASFLYSGVPSRFSGSRSGTDFTLTISSLQPED |
| | | | FATYYCQQHYTTPPTFGQGTKVEIKRTVAAPSVFIFPPSDERLKS |
| | | | GTASVVCLLNNFYPREAKVQWKVDNALQSGNSKESVTEQDSKDST |
| | | | YSLSSRLTLSKADYEKHKVYACEVTHQGLSSPVTKSFNRGEC |
| 16 | 5037 | VL | DIQMTQSPSSLSASVGDRVTITCRASQDVNTAVAWYQQKPGKAPK |
| | | | LLIYSASFLYSGVPSRFSGSRSGTDFTLTISSLQPEDFATYYCQQ |
| | | | HYTTPPTFGQGTKVEIK |
| 17 | 5037 | L1 | QDVNTA |
| 18 | 5037 | L3 | QQHYTTPPT |
| 19 | 5037 | L2 | SAS |
| 20 | 3382 | Full | GDIQMTQSPSSLSASVGDRVTITCKASQDVSIGVAWYQQKPGKAP |
| | | | KLLIYSASYRYTGVPSRFSGSGSGTDFTLTISSLQPEDFATYYCQ |
| | | | QYYIYPATFGQGTKVEIKRTVAAPSVFIFPPSDEQLKSGTASVVC |
| | | | LLNNFYPREAKVQWKVDNALQSGNSQESVTEQDSKDSTYSLSSTL |
| | | | TLSKADYEKHKVYACEVTHQGLSSPVTKSFNRGEC |
| 21 | 3382 | VL | DIQMTQSPSSLSASVGDRVTITCKASQDVSIGVAWYQQKPGKAPK |
| | | | LLIYSASYRYTGVPSRFSGSGSGTDFTLTISSLQPEDFATYYCQQ |
| | | | YYIYPATFGQGTKVEIK |
| 22 | 3382 | L1 | QDVSIG |
| 23 | 3382 | L3 | QQYYIYPAT |
| 24 | 3382 | L2 | SAS |
| 25 | 5065 | Full | EVQLVESGGGLVQPGGSLRLSCAASGFNIKDTYIHWVRQAPGKGL |
| | | | EWVARIYPTNGYTRYADSVKGRFTISADTSKNTAYLQMNSLRAED |
| | | | TAVYYCSRWGGDGFYAMDYWGQGTLVTVSSASTKGPSVFPLAPSS |
| | | | KSTSGGTAALGCEVTDYFPEPVTVSWNSGALTSGVHTFPAVLQSS |
| | | | GLYSLSSVVTVPSSSLGTQTYICNVNHKPSNTKVDKKVEPKSCDK |
| | | | THTCPPCPAPELLGGPSVFLFPPKPKDTLMISRTPEVTCVVVDVS |
| | | | HEDPEVKFNWYVDGVEVHNAKTKPREEQYNSTYRVVSVLTVLHQD |
| | | | WLNGKEYKCKVSNKALPAPIEKTISKAKGQPREPQVYVYPPSRDE |
| | | | LTKNQVSLTCLVKGFYPSDIAVEWESNGQPENNYKTTPPVLDSDG |
| | | | SFALVSKLTVDKSRWQQGNVFSCSVMHEALHNHYTQKSLSLSPG |
| 26 | 5065 | VH | EVQLVESGGGLVQPGGSLRLSCAASGFNIKDTYIHWVRQAPGKGL |
| | | | EWVARIYPTNGYTRYADSVKGRFTISADTSKNTAYLQMNSLRAED |
| | | | TAVYYCSRWGGDGFYAMDYWGQGTLVTVSS |
| 27 | 5065, | H1 | GFNIKDTY |
| | 720, | | |
| | 719 | | |
| 28 | 5065, | H3 | SRWGGDGFYAMDY |
| | 720, | | |
| | 719 | | |
| 29 | 5065, | H2 | IYPTNGYT |
| | 720, | | |
| | 719 | | |
| 30 | 6586 | Full | GEVQLVESGGGLVQPGGSLRLSCAASGFTFADYTMDWVRQAPGKG |
| | | | LEWVGDVNPNSGGSIYNQRFKGRFTFSVDRSKNTLYLQMNSLRAE |
| | | | DTAVYYCARNLGPSFYFDYWGQGTLVTVSSASTKGPSVFPLAPSS |
| | | | KSTSGGTAALGCLVKDYFPEPVTVSWNSGALTSGVHTFPAVLQSS |
| | | | GLYSLSSVVTVPSSSLGTQTYICNVNHKPSNTKVDKKVEPKSCDK |
| | | | THTCPPCPAPELLGGPSVFLFPPKPKDTLMISRTPEVTCVVVDVS |
| | | | HEDPEVKFNWYVDGVEVHNAKTKPREEQYNSTYRVVSVLTVLHQD |
| | | | WLNGKEYKCKVSNKALPAPIEKTISKAKGQPREPQVYVYPPSRDE |
| | | | LTKNQVSLTCLVKGFYPSDIAVEWESNGQPENNYKTTPPVLDSDG |
| | | | SFALVSKLTVDKSRWQQGNVFSCSVMHEALHNHYTQKSLSLSPG |
| 31 | 6586 | VH | EVQLVESGGGLVQPGGSLRLSCAASGFTFADYTMDWVRQAPGKGL |
| | | | EWVGDVNPNSGGSIYNQRFKGRFTFSVDRSKNTLYLQMNSLRAED |
| | | | TAVYYCARNLGPSFYFDYWGQGTLVTVSS |
| 32 | 6586 | H1 | GFTFADYT |
| 33 | 6586 | H3 | ARNLGPSFYFDY |
| 34 | 6586 | H2 | VNPNSGGS |
| 35 | 3904 | Full | YPYDVPDYATGSDIQMTQSPSSLSASVGDRVTITCKASQDVSIGV |
| | | | AWYQQKPGKAPKLLIYSASYRYTGVPSRFSGSGSGTDFTLTISSL |
| | | | QPEDFATYYCQQYYIYPYTFGQGTKVEIKRTVAAPSVFIFPPSDE |
| | | | ELKSGTASVVCLLNNFYPREAKVQWKVDNALQSGNSEESVTEQDS |
| | | | KDSTYSLSSTLELSKADYEKHKVYACEVTHQGLSSPVTKSFNRGE |
| | | | C |
| 36 | 3904 | VL | DIQMTQSPSSLSASVGDRVTITCKASQDVSIGVAWYQQKPGKAPK |
| | | | LLIYSASYRYTGVPSRFSGSGSGTDFTLTISSLQPEDFATYYCQQ |
| | | | YYIYPYTFGQGTKVEIK |
| 37 | 719 | Full | DIQMTQSPSSLSASVGDRVTITCRASQDVNTAVAWYQQKPGKAPK |
| | | | LLIYSASFLYSGVPSRFSGSRSGTDFTLTISSLQPEDFATYYCQQ |
| | | | HYTTPPTFGQGTKVEIKGGSGGGSGGGSGGGSGGGSGEVQLVESG |
| | | | GGLVQPGGSLRLSCAASGFNIKDTYIHWVRQAPGKGLEWVARIYP |
| | | | TNGYTRYADSVKGRFTISADTSKNTAYLQMNSLRAEDTAVYYCSR |
| | | | WGGDGFYAMDYWGQGTLVTVSSAAEPKSSDKTHTCPPCPAPELLG |
| | | | GPSVFLFPPKPKDTLMISRTPEVTCVVVDVSHEDPEVKFNWYVDG |
| | | | VEVHNAKTKPREEQYNSTYRVVSVLTVLHQDWLNGKEYKCKVSNK |
| | | | ALPAPIEKTISKAKGQPREPQVYTYPPSRDELTKNQVSLTCLVKG |
| | | | FYPSDIAVEWESNGQPENNYKTTPPVLDEDGSFALVSKLTVDKSR |
| | | | WQQGNVFSCSVMHEALHNHYTQKSLSLSPGK |
| 38 | 719 | VL | DIQMTQSPSSLSASVGDRVTITCRASQDVNTAVAWYQQKPGKAPK |
| | | | LLIYSASFLYSGVPSRFSGSRSGTDFTLTISSLQPEDFATYYCQQ |
| | | | HYTTPPTFGQGTKVEIK |
| 39 | 719 | VH | EVQLVESGGGLVQPGGSLRLSCAASGFNIKDTYIHWVRQAPGKGL |
| | | | EWVARIYPTNGYTRYADSVKGRFTISADTSKNTAYLQMNSLRAED |
| | | | TAVYYCSRWGGDGFYAMDYWGQGTLVTVSS |
| 40 | 720 | Full | DIQMTQSPSSLSASVGDRVTITCRASQDVNTAVAWYQQKPGKAPK |
| | | | LLIYSASFLYSGVPSRFSGSRSGTDFTLTISSLQPEDFATYYCQQ |
| | | | HYTTPPTFGQGTKVEIKGGSGGGSGGGSGGGSGGGSGEVQLVESG |
| | | | GGLVQPGGSLRLSCAASGFNIKDTYIHWVRQAPGKGLEWVARIYP |
| | | | TNGYTRYADSVKGRFTISADTSKNTAYLQMNSLRAEDTAVYYCSR |
| | | | WGGDGFYAMDYWGQGTLVTVSSAAEPKSSDKTHTCPPCPAPELLG |
| | | | GPSVFLFPPKPKDTLMISRTPEVTCVVVDVSHEDPEVKFNWYVDG |
| | | | VEVHNAKTKPREEQYNSTYRVVSVLTVLHQDWLNGKEYKCKVSNK |
| | | | ALPAPIEKTISKAKGQPREPQVYTLPPSRDELTKNQVSLICLVKG |
| | | | FYPSDIAVEWESNGQPENRYMTWPPVLDSDGSFFLYSKLTVDKSR |
| | | | WQQGNVFSCSVMHEALHNHYTQKSLSLSPGK |
| 41 | 720 | VL | DIQMTQSPSSLSASVGDRVTITCRASQDVNTAVAWYQQKPGKAPK |
| | | | LLIYSASFLYSGVPSRFSGSRSGTDFTLTISSLQPEDFATYYCQQ |
| | | | HYTTPPTFGQGTKVEIK |
| 42 | 720 | VH | EVQLVESGGGLVQPGGSLRLSCAASGFNIKDTYIHWVRQAPGKGL |
| | | | EWVARIYPTNGYTRYADSVKGRFTISADTSKNTAYLQMNSLRAED |
| | | | TAVYYCSRWGGDGFYAMDYWGQGTLVTVSS |
| 43 | 3041 | Full | EVQLVESGGGLVQPGGSLRLSCAASGFTFTDYTMDWVRQAPGKGL |
| | | | EWVADVNPNSGGSIYNQRFKGRFTLSVDRSKNTLYLQMNSLRAED |
| | | | TAVYYCARNLGPSFYFDYWGQGTLVTVSSASTKGPSVFPLAPSSK |
| | | | STSGGTAALGCLVKDYFPEPVTVSWNSGALTSGVHTFPAVLQSSG |
| | | | LYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSH |
| | | | EDPEVKFNWYVDGVEVHNAKTKPREEQYNSTYRVVSVLTVLHQDW |
| | | | LNGKEYKCKVSNKALPAPIEKTISKAKGQPREPQVYVLPPSRDEL |
| | | | TKNQVSLLCLVKGFYPSDIAVEWESNGQPENNYLTWPPVLDSDGS |
| | | | FFLYSKLTVDKSRWQQGNVFSCSVMHEALHNHYTQKSLSLSPG |
| 44 | 3041 | VH | EVQLVESGGGLVQPGGSLRLSCAASGFTFTDYTMDWVRQAPGKGL |
| | | | EWVADVNPNSGGSIYNQRFKGRFTLSVDRSKNTLYLQMNSLRAED |
| | | | TAVYYCARNLGPSFYFDYWGQGTLVTVSS |
| 45 | 3057 | Full | EVQLVESGGGLVQPGGSLRLSCAASGFTFTDYTMDWVRQAPGKGL |
| | | | EWVADVNPNSGGSIYNQRFKGRFTLSVDRSKNTLYLQMNSLRAED |
| | | | TAVYYCARNLGPSFYFDYWGQGTLVTVSSASTKGPSVFPLAPSSK |
| | | | STSGGTAALGCLVKDYFPEPVTVSWNSGALTSGVHTFPAVLQSSG |
| | | | LYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSH |
| | | | EDPEVKFNWYVDGVEVHNAKTKPREEQYNSTYRVVSVLTVLHQDW |
| | | | LNGKEYKCKVSNKALPAPIEKTISKAKGQPREPQVYVYPPSRDEL |
| | | | TKNQVSLTCLVKGFYPSDIAVEWESNGQPENNYKTTPPVLDSDGS |
| | | | FALVSKLTVDKSRWQQGNVFSCSVMHEALHNHYTQKSLSLSPG |
| 46 | 3057 | VH | EVQLVESGGGLVQPGGSLRLSCAASGFTFTDYTMDWVRQAPGKGL |
| | | | EWVADVNPNSGGSIYNQRFKGRFTLSVDRSKNTLYLQMNSLRAED |
| | | | TAVYYCARNLGPSFYFDYWGQGTLVTVSS |
| 47 | 3317 | Full | DIQMTQSPSSLSASVGDRVTITCKASQDVSIGVAWYQQKPGKAPK |
| | | | LLIYSASYRYTGVPSRFSGSGSGTDFTLTISSLQPEDFATYYCQQ |
| | | | YYIYPYTFGQGTKVEIKGGGGSGGGGSGGGGSEVQLVESGGGLVQ |
| | | | PGGSLRLSCAASGFTFTDYTMDWVRQAPGKGLEWVADVNPNSGGS |
| | | | IYNQRFKGRFTLSVDRSKNTLYLQMNSLRAEDTAVYYCARNLGPS |
| | | | FYFDYWGQGTLVTVSSAAEPKSSDKTHTCPPCPAPELLGGPSVFL |
| | | | FPPKPKDTLMISRTPEVTCVVVDVSHEDPEVKFNWYVDGVEVHNA |
| | | | KTKPREEQYNSTYRVVSVLTVLHQDWLNGKEYKCKVSNKALPAPI |
| | | | EKTISKAKGQPREPQVYVYPPSRDELTKNQVSLTCLVKGFYPSDI |
| | | | AVEWESNGQPENNYKTTPPVLDSDGSFALVSKLTVDKSRWQQGNV |
| | | | FSCSVMHEALHNHYTQKSLSLSPGK |
| 48 | 3317 | VL | DIQMTQSPSSLSASVGDRVTITCKASQDVSIGVAWYQQKPGKAPK |
| | | | LLIYSASYRYTGVPSRFSGSGSGTDFTLTISSLQPEDFATYYCQQ |
| | | | YYIYPYTFGQGTKVEIK |
| 49 | 3317 | VH | EVQLVESGGGLVQPGGSLRLSCAASGFTFTDYTMDWVRQAPGKGL |
| | | | EWVADVNPNSGGSIYNQRFKGRFTLSVDRSKNTLYLQMNSLRAED |
| | | | TAVYYCARNLGPSFYFDYWGQGTLVTVSS |
| 50 | 5244 | Full | GDIQMTQSPSSLSASVGDRVTITCRASQDVNTAVAWYQQKPGKAP |
| | | | KLLIYSASFLYSGVPSRFSGSRSGTDFTLTISSLQPEDFATYYCQ |
| | | | QHYTTPPTFGQGTKVEIKGGSGGGSGGGSGGGSGGGSGEVQLVES |
| | | | GGGLVQPGGSLRLSCAASGFNIKDTYIHWVRQAPGKGLEWVARIY |
| | | | PTNGYTRYADSVKGRFTISADTSKNTAYLQMNSLRAEDTAVYYCS |
| | | | RWGGDGFYAMDYWGQGTLVTVSSAAEPKSSDKTHTCPPCPAPELL |
| | | | GGPSVFLFPPKPKDTLMISRTPEVTCVVVDVSHEDPEVKFNWYVD |
| | | | GVEVHNAKTKPREEQYNSTYRVVSVLTVLHQDWLNGKEYKCKVSN |
| | | | KALPAPIEKTISKAKGQPREPQVYVLPPSRDELTKNQVSLLCLVK |
| | | | GFYPSDIAVEWESNGQPENNYLTWPPVLDSDGSFFLYSKLTVDKS |
| | | | RWQQGNVFSCSVMHEALHNHYTQKSLSLSPG |
| 51 | 5244 | VL | DIQMTQSPSSLSASVGDRVTITCRASQDVNTAVAWYQQKPGKAPK |
| | | | LLIYSASFLYSGVPSRFSGSRSGTDFTLTISSLQPEDFATYYCQQ |
| | | | HYTTPPTFGQGTKVEIK |
| 52 | 5244 | VH | EVQLVESGGGLVQPGGSLRLSCAASGFNIKDTYIHWVRQAPGKGL |
| | | | EWVARIYPTNGYTRYADSVKGRFTISADTSKNTAYLQMNSLRAED |
| | | | TAVYYCSRWGGDGFYAMDYWGQGTLVTVSS |
| 53 | 5244, | L1 | QDVNTA |
| | 5034, | | |
| | 719, | | |
| | 720 | | |
| 54 | 5244, | L2 | SAS |
| | 5034, | | |
| | 719, | | |
| | 720 | | |
| 55 | 5244, | L3 | QQHYTTPPT |
| | 5034, | | |
| | 719, | | |
| | 720 | | |
| 56 | 5244 | H1 | GFNIKDTY |
| 57 | 5244 | H2 | IYPTNGYT |
| 58 | 5244 | H3 | SRWGGDGFYAMDY |

1. A method of treating a subject having a HER2-expressing cancer comprising administering to the subject an effective amount of an anti-HER2 biparatopic antibody, wherein the effective amount is administered according to a 2-tiered fixed dosing regimen comprising administering, at a fixed time interval, a low fixed dose to a subject whose weight is less than a dose cut-off weight, and a high fixed dose to a subject whose weight is equal to or greater than the dose cut-off weight.

2. The method according to claim 1, wherein the low fixed dose is about 600 mg and the high fixed dose is about 800 mg, the dose cut-off weight is 70 kg and the fixed time interval is weekly (QW).

3. The method according to claim 1, wherein the low fixed dose is about 800 mg and the high fixed dose is about 1200 mg, the dose cut-off weight is 70 kg and the fixed time interval is weekly (QW).

4. The method according to claim 1, wherein the low fixed dose is about 800 mg and the high fixed dose is about 1000 mg, the dose cut-off weight is 70 kg and the fixed interval is weekly (QW).

5. The method according to claim 1, wherein the low fixed dose is about 1800 mg and the high fixed dose is about 2200 mg, the dose cut-off weight is 70 kg and the fixed time interval is every 2 weeks (Q2W).

6. The method according to claim 1, wherein the low fixed dose is about 1200 mg and the high fixed dose is about 1600 mg, the dose cut-off weight is 70 kg and the fixed time interval is every 2 weeks (Q2W).

7. The method according to claim 1, wherein the low fixed dose is about 1200 mg and the high fixed dose is about 1800 mg, the dose cut-off weight is 70 kg and the fixed time interval is every 3 weeks (Q3W).

8. The method according to claim 1, wherein the low fixed dose is about 1800 mg and the high fixed dose is about 2400 mg, the dose cut-off weight is 70 kg and the fixed time interval is every 3 weeks (Q3W).

9. The method according to claim 1, wherein the anti-HER2 biparatopic antibody comprises (a) a first antigen-binding domain comprising CDR sequences CDRH1, CDRH2 and CDRH3 as set forth in SEQ ID NOs: 32, 34 and 33 respectively, and CDR sequences CDRL1, CDRL2 and CDRL3 as set forth in SEQ ID NOs: 22, 24 and 23 respectively, and (b) a second antigen-binding domain comprising CDR sequences CDRH1, CDRH2 and CDRH3 as set forth in SEQ ID NOs: 56, 57 and 58 respectively, and CDR sequences CDRL1, CDRL2 and CDRL3 sequences as set forth in SEQ ID NOs: 53, 54 and 55 respectively.

10. The method according to claim 9, wherein the first antigen-binding domain is a Fab and the second antigen-binding domain is an scFv.

11. The method according to claim 9, wherein the anti-HER2 biparatopic antibody comprises (a) a first antigen-binding domain comprising a variable heavy chain region (VH) comprising the sequence as set forth in SEQ ID NO: 31, and a variable light chain region (VL) comprising the sequence as set forth in SEQ ID NO: 21, and (b) a second antigen-binding domain comprising a VH sequence as set forth in SEQ ID NO: 52, and a VL sequence as set forth in SEQ ID NO: 51.

12. The method according to claim 1, wherein the anti-HER2 biparatopic antibody comprises a heavy chain H1 comprising the sequences set forth in SEQ ID NO: 30, and heavy chain H2 comprising the sequences set forth in SEQ ID NO: 50 and a light chain L1 comprising the sequences set forth in SEQ ID NO: 20.

13. The method according to claim 1, wherein the HER2-expressing cancer is a solid tumor.

14. The method according to claim 1, wherein the HER2-expressing cancer is breast cancer, biliary tract cancer, gastroesophageal adenocarcinoma (GEA), gastroesophageal esophageal junction cancer (GEJ), esophageal cancer, gastric cancer, endometrial cancer, ovarian cancer, cervical cancer, non-small cell lung cancer (NSCLC), anal cancer or colorectal cancer (CRC).

15. The method according to claim 1, wherein the HER2-expressing cancer is gastroesophageal adenocarcinoma (GEA).

16. The method according to claim 1, wherein the subject has received prior treatment with one or more of the anti-HER2-targeted therapies trastuzumab, pertuzumab, T-DM1 or Enhertu™ (fam-trastuzumab deruxtecan-nxki).

17. The method according to claim 1, wherein the subject has not received prior treatment with an anti-HER2 targeted therapy.

18. (canceled)

19. The method according to claim 1, wherein the cancer is metastatic or locally advanced.

20. (canceled)

21. The method according to claim 1, wherein the cancer is HER2 3+, HER2 2+/3+, HER2 2+ or HER2 1+.

22-24. (canceled)

25. The method according to claim 1, wherein the biparatopic antibody is administered in combination with one or more chemotherapy regimens.

26. The method according to claim 25 wherein the chemotherapy regimen comprises mFOLFOX6 (fluorouracil 5-FU) and leucovorin plus oxaliplatin), CAPOX (capecitabine plus oxaliplatin) or FP (fluorouracil (5-FU) plus cisplatin).

27. The method according to claim 25 wherein the chemotherapy regimen comprises a taxane.

28. The method according to claim 1, wherein the anti-HER2 biparatopic antibody is administered in combination with a PD-1 inhibitor or another anti-HER2 agent.

29. (canceled)

30. The method according to claim 28, wherein the PD-1 inhibitor is an anti-PD-1 antibody.

31. The method according to claim 30, wherein the anti-PD-1 antibody is selected from tislelizumab, pembrolizumab, nivolumab or cemiplimab.

32. (canceled)

33. A method of treating a HER2-expression cancer comprising administering an anti-HER2 biparatopic antibody to a subject, wherein the effective dose of the antibody is a two-tiered fixed dose regimen comprising a low fixed dose for a subject whose weight is less than a dose cut-off weight, and a high fixed dose for a subject whose weight is greater than or equal to a dose cut-off weight, wherein the HER-2 expressing cancer is breast cancer, gastroesophageal adenocarcinoma (GEA), esophageal cancer, gastroesophageal junction cancer (GEJ), gastric cancer, endometrial cancer, ovarian cancer, cervical cancer, non-small cell lung cancer (NSCLC), anal cancer or colorectal cancer (CRC), and wherein the subject has received prior treatment with one or more anti-HER2-targeted therapies.

34. The antibody according to claim 33, wherein the low fixed dose is about 800 mg and the high fixed dose is about 1200 mg, the dose cut-off weight is 70 kg and the fixed time internal is weekly (QW).

35. The antibody according to claim 33, wherein the low fixed dose is about 1200 mg and the high fixed dose is about 1600 mg, the dose cut-off weight is 70 kg and the fixed time internal is every 2 weeks (Q2W).

36. The antibody according to claim 33, wherein the low fixed dose is about 1800 mg and the high fixed dose is about 2400 mg, the dose cut-off weight is 70 kg and the fixed time internal is every 3 weeks (Q3W).

37. The antibody according claim 33 wherein the antibody is v10000.

38. A pharmaceutical kit comprising: (i) one or more containers comprising an anti-HER-2 biparatopic antibody and (ii) a label or package insert in or associated with the one or more containers indicating that the anti-HER2 biparatopic antibody is for administration to a subject having a HER2-expressing cancer (a) at a dose of 1800 mg for a subject weighting less than 70 kg or (b) at a dose of 2400 mg for a subject weighing 70 kg or more, administered every 3 weeks (Q3W).

39. A pharmaceutical kit comprising: (i) one or more containers comprising an anti-HER-2 biparatopic antibody and (ii) a label or package insert in or associated with the one or more containers indicating that the anti-HER2 biparatopic antibody is for administration to a subject having a HER2-expressing cancer (a) at a dose of 1200 mg for a subject weighting less than 70 kg or (b) at a dose of 1600 mg for a subject weighing 70 kg or more, administered every 2 weeks (Q2W).

40-47. (canceled)

48. The method according to claim 1, wherein the cancer comprises HER2 gene amplification.

49. The method according to claim 1, wherein the subject has not received prior systemic treatment with a chemotherapeutic agent.

50. The method according to claim 26, wherein the mFOLFOX6 is administered as
leucovorin 400 mg/m² intravenously (IV), and oxaliplatin 85 mg/m² IV every 2 weeks (Q2W) on day 1 and day 15 of each 28-day cycle; and
5-FU 1200 mg/m² IV continuous infusion on each day for a total of 2400 mg/m² over 46 to 48 hours Q2W on days 1-2 and days 15-16 of each 28-day cycle.

51. The method according to claim 26, wherein the CAPOX is administered as
capecitabine at 1,000 mg/m² orally (PO) twice a day (BID) on Days 1-14 of each 21-day cycle; and
oxaliplatin at 130 mg/m² IV Day 1 of each 21-day cycle.

52. The method according to claim 26, wherein the FP is administered as
cisplatin at 80 mg/m² IV Q3W on day 1 of each 21 day cycle; and
5-FU at 800 mg/m²/day IV on days 1-5 of each 21-day cycle.
